(19) United States
(12) Patent Application Publication
(10) Pub. No.: US 2024/0409669 A1
(43) Pub. Date: Dec. 12, 2024

(54) Neutralizing Trivalent Protein-DNA Molecules (TRI-PDBODY) for SARS-COV-2 Infection Treatment

(71) Applicant: ARIZONA BOARD OF REGENTS ON BEHALF OF ARIZONA STATE UNIVERSITY, Scottsdale, AZ (US)

(72) Inventors: Nicholas Stephanopoulos, Scottsdale, AZ (US); Yang Xu, Chandler, AZ (US); Petr Sulc, Scottsdale, AZ (US); Hao Yan, Gilbert, AZ (US); Rong Zheng, Tempe, AZ (US); Abhay Prasad, Tempe, AZ (US)

(21) Appl. No.: 18/701,901

(22) PCT Filed: Oct. 20, 2022
(86) PCT No.: PCT/US2022/078434

§ 371 Date: Apr. 16, 2024

Related U.S. Application Data
(60) Provisional application No. 63/257,832, filed on Oct. 20, 2021.

(51) Int. Cl. C07K 19/00; A61K 38/00; A61P 31/14; C07K 14/005

(52) U.S. Cl. CPC C07K 19/00; A61P 31/14; C07K 14/005; A61K 38/00; C07K 2319/30

(57) ABSTRACT

The present disclosure relates to DNA-peptide hybrid molecules. In some embodiments, the DNA-peptide hybrid molecules comprise target-specific binding peptides which selectively bind to SARS-COV-2 surface glycoprotein. Methods of using DNA-peptide hybrid molecules in the treatment of SARS-COV-2 infection are also provided.

### CROSS-REFERENCE TO RELATED APPLICATIONS

The present application claims priority to U.S. Provisional Patent Application No. 63/257,832 filed Oct. 20, 2021, the entire contents of which are hereby incorporated by reference.

### REFERENCE TO AN ELECTRONIC SEQUENCE LISTING

The contents of the electronic sequence listing (112624.01371.xml; Size: 7,023 bytes; and Date of Creation: Oct. 14, 2022) is herein incorporated by reference in its entirety.

### STATEMENT REGARDING FEDERALLY SPONSORED RESEARCH

This invention was made with government support under GM132931 awarded by the National Institutes of Health. The government has certain rights in the invention.

### FIELD OF INVENTION

The present disclosure generally relates to DNA-peptide hybrid nanostructures. In some embodiments, the DNA-peptide hybrid molecules specifically bind to a target of interest. Also disclosed are methods of using DNA-peptide hybrid molecules in the treatment of a disease or disorder.

### BACKGROUND

The ability to block protein-protein interactions (PPIs) is crucial not just for therapeutic purposes—e.g. neutralizing antibodies for pathogenic threats like SARS-COV-2, or small molecule drugs for cancer therapy—but also for fundamental biological studies. Countless biological processes are mediated by protein-protein interactions, such as cell-cell interactions, signal transduction, cell-matrix interactions, immune system recognition, and many others, but it can be difficult to block these interactions with high affinity and specificity. Approaches like small molecule drugs, or peptides found through rational design or high-throughput evolutionary methods like phage, mRNA, or ribosome display are often hindered by lack of binding to the key protein-protein interface. Antibodies can block PPIs, but again must target a key interface. Furthermore, these methods are generally not reversible or triggerable, and cannot be switched “on” and “off” on-demand with simple triggers. Therefore, there is a need in the art for reversible or triggerable molecules that are able to bind to proteins and inhibit protein-protein interactions.

### SUMMARY

In a first aspect of the current disclosure, DNA-peptide hybrid molecules are provided. In some embodiments, the DNA-peptide hybrid molecules comprise a DNA nanostructure chemically linked to one or more target-specific binding peptides, wherein the one or more target-specific binding peptides are specific for SARS-COV-2. In some embodiments, the DNA nanostructure is selected from the group consisting of: a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool. In some embodiments, the DNA nanostructure comprises one of a three-helix bundle or a four-helix bundle. In some embodiments, the target-specific binding peptide is LCB1. In some embodiments, the distance between the target-specific binding peptides on the fully assembled DNA-peptide hybrid molecule is about the distance between the sites where the target-specific binding peptide binds to the native SARS-COV-2 homotrimeric spike protein. In some embodiments, the DNA-peptide hybrid molecule has greater affinity for SARS-COV-2 spike protein compared to the one or more target-specific binding peptides alone. In some embodiments, the DNA-peptide hybrid molecule has a K_{d} of less than about 5 nM for a spike protein or variant, such as but no limited to each of the spike protein of SARS-COV-2 alpha, beta, gamma, and delta variants. In some embodiments, the DNA-peptide hybrid molecule has a K_{d} of less than about 1 nM for a spike protein variant such as but not limited to each of the spike protein of SARS-COV-2 alpha, beta, gamma, and delta variants. In some embodiments, the synthetic DNA-peptide hybrid molecule further comprises an immunoglobulin Fc domain. In some embodiments, binding of the DNA-peptide hybrid molecule to SARS-COV-2 spike protein induces immune cells to engage in antibody dependent cellular cytotoxicity (ADCC). In some embodiments, the one or more target-specific binding peptides and the DNA nanostructure are linked through a disulfide bond or through copper-free click chemistry between a protein bearing the non-canonical amino acid 4-azidophenylalanine and DNA linked to cyclooctyne. In some embodiments, the chemical linkage of the one or more target-specific binding peptides to the DNA nanostructure is cleavable. In some embodiments, the chemical linkage comprises a photocleavable linkage.

In a second aspect of the current disclosure, methods of treating a subject in need thereof are provided. In some embodiments of the methods, the method comprises administering to the subject a DNA-peptide hybrid molecule comprising a DNA nanostructure chemically linked to one or more target-specific binding peptides, wherein the one or more target-specific binding peptides are specific for SARS-COV-2, in an amount sufficient to treat SARS-COV-2 infection. In some embodiments of the methods, the DNA nanostructure is selected from the group consisting of: a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool. In some embodiments of the methods, the DNA nanostructure comprises one of a three-helix bundle or a four-helix bundle. In some embodiments of the methods, the one or more target-specific binding peptides is LCB1.

In some embodiments the distance between the target-specific binding peptides on the fully assembled DNA-peptide hybrid molecule is about the distance between the sites where the target-specific binding peptide binds to the native SARS-COV-2 homotrimeric spike protein. In some embodiments of the methods, the DNA-peptide hybrid molecule has greater affinity for SARS-COV-2 spike protein than the target-specific peptide alone. In some embodiments of the methods, the DNA-peptide hybrid molecule has a K_{d} of less than about 5 nM for a spike protein or a variant, such as but not limited to each of the spike protein of SARS-COV-2 alpha, beta, gamma, and delta variants. In some embodiments of the methods, the DNA-peptide hybrid molecule further comprises an immunoglobulin Fc domain. In some embodiments of the methods, binding of the DNA-peptide hybrid molecule to SARS-COV-2 spike protein induces immune cells to engage in antibody dependent cellular cytotoxicity (ADCC) and antibody dependent cellular phagocytosis (ADCP). In some embodiments of the methods, the one or more target-specific binding peptides and the DNA nanostructure are linked through a disulfide bond or through copper-free click chemistry between a peptide bearing the non-canonical amino acid 4-azidophenylalanine and DNA linked to cyclooctyne. In some embodiments of the methods, the chemical linkage of the one or more target-specific binding peptides to the DNA nanostructure is cleavable. In some embodiments of the methods, the chemical linkage comprises a photocleavable linkage. In some embodiments of the methods, the subject in need thereof is infected with SARS-CoV-2. In some embodiments of the methods, the subject in need thereof is not infected with SARS-COV-2. In some embodiments of the methods, the DNA-peptide hybrid molecule is administered as a pharmaceutical composition comprising one or more suitable excipients, solvents, or vehicles whereby the pharmaceutical composition can be effectively administered to a subject in need thereof.

In some embodiments of the compositions and methods, the DNA-peptide hybrid molecules are used to detect SARS-COV-2. In some embodiments, the DNA-peptide hybrid molecules are linked to a solid support or are linked to a detectable label.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1: Overview of approach. Panel A) shows nanobodies bind to a target (the SARS-COV-2 spike protein trimer here) through three CDR loops (yellow, blue, green), but must target the key ACE2 binding interface to block infection. Panel B) shows a programmable DNA nanostructure that positions three protein or peptide ligands can block any protein without having to bind to the key interface directly.

FIG. 2: Protein bioconjugation chemistry and preliminary data. Proteins can be conjugated to DNA (red sphere) using either cysteine based chemistry shown in Panel (A) or copper-free click chemistry with 4-azidophenylalanine-containing proteins shown in Panel (B). Panel C) shows MALDI-TOF mass spectrum of a peptide-DNA conjugate with an RBD-binding sequence P1. The DNA sequence is SEQ ID NO: 4 and the P1 protein sequence is SEQ ID NO: 5. Panel D) shows ELISA assay: LCB1 protein was immobilized on a surface and exposed to increasing concentrations of the spike S1 (RBD-containing) protein, followed by a primary antibody and a secondary antibody-HRP conjugate. Competition with excess free LCB1 abrogated the interaction. Panel E) shows surface plasmon resonance (SPR) analysis of monomeric spike RBD on a surface exposed to LCB1 in solution.

FIG. 3: Nanostructures used. Panel A) shows triangular DNA origami with handles for capturing a homotrivalent protein-DNA conjugate in the central cavity shown in panel (B). Panel C) shows native PAGE of three different DNA nanostructures: a 3-way junction, 6-helix bundle, and tetrahedral cage. All three can be annealed at high yield and purify. Panel D) shows hybrid protein-DNA cage from protein-DNA conjugates. Panel E) shows block-like DNA origami cuboid with addressable faces. F) Reconfiguarable DNA nano-tweezer with tunable arm lengths and distances between them. Panels (G-L) show proposed nano-scaffold designs for DNA-peptide hybrid molecules: four-helix bundle (G), six-helix bundle (H), two variants of a three-way junction (I, J), tetrahedral cage (K), and three-legged “stool” (L). All structures are scaled to roughly the same dimensions (with 5 nm scale indicated). Proteins and peptides can be attached to the ends of all helices, as well as nick points in the sides of the tetrahedral cages (indicated by red asterisks in (K)).

FIG. 4: In silico nanostructure evolution. A starting design, with its three peptide attachment sites colored in red, green and blue, is mutated by introducing a single-stranded region (pink) with 3 Tbases. The mean structures obtained from oxDNA simulation show that the distances between the peptide-functionalized sites change by several nanometers.

FIG. 5: Homo-trivalent LCB1 DNA-peptide hybrid molecules. A) To-scale model of a 4-helix bundle with three copies of LCB1 binding to the spike protein trimer (4HB-LCB1). B) AFM image of 4HB-LCB1. C) Inhibition of spike protein monomer binding to immobilized LCB1; both free LCB1 and its ssDNA conjugate inhibit equally. D) The trivalent 4HB-LCB1 DNA-peptide hybrid molecule is more effective at inhibiting RBD binding than monomeric LCB1. E) Heat map showing how far (blue: closer, red: farther) one arm (red arrow) of a DNA-peptide hybrid molecule can reach when another arm (green arrow) is bound to a known site. F) Computationally predicted binding sites for the nanobody that targets the spike NTD. Red arrows indicate spurious predictions, the green arrow indicates the known, correct NTD site.

FIG. 6: DNA-peptide hybrid molecule photocleavage. Attaching the protein ligands to the DNA-peptide hybrid molecule via DNA with photocleavable linkers will allow for removal of the nanostructure—and restoration of the protein-protein interaction—upon exposure to UV light.

FIG. 7: “CLASP” system. Panel A) shows standard IgG antibody structure with variable heavy (VH) region comprised of three CDRs. Panel B) shows CDR3 native conformation compared to the cyclized constrained CDR 3 peptide (the CLASP system). “B” denotes a bioconjugation handle, e.g. an alkyne for click.

FIG. 8: Validation of temporally sensitive TBI CLASPs. Panels A-D show qualitative representation of acute TBI CLASP (green) and cell nuclei (blue) on 1 dpi mouse CCI tissue (A, B), sham control mouse tissue (C) or 7 dpi CCI tissue (D). Panel E shows subacute TBI CLASP staining on 7 dpi mouse CCI tissue.

FIG. 9: Likely binding sites for the known fibrinogen (PDB: Ifza) binding peptide GPRPXX (SEQ ID NO: 3) obtained from global docking software GalaxyPepdock. Nanorulers will be designed to connect candidate sites for CD3 peptides from phage display experiments and this validated GPRPXX (SEQ ID NO: 3) binding pocket.

FIG. 10: Mini-binder binding avidity by ELISA.

FIG. 11: Mini-binder neutralization assay.

FIG. 12: Mini-binder binding with spike trimer equivalent constants by surface plasmon resonance.

FIG. 13: 4HB-Lcb triPDbody assemble and competitive inhibition assay.

FIG. 14: To evaluate the binding affinity (KD) of LCB for spike variants by SP.

FIG. 15: Variant of Concern (VOC) binding affinity with mini-binder.

FIG. 16: Tri-pdbody (Trivalent mini-binder 4HB) overview.

FIG. 17: Overview of the trivalent pdbody mediated ADCC and ADCP Immunol effect.

FIG. 18: Overview of the tri-valent DNA-protein nanostructure. Panel (A) shows molecular surface representation of mini-binder bound to the SARS-COV-2 spike ectodomain trimer viewed along two orthogonal axes (left, side view; right, top view). Panel (B) shows wild type mini-binder and Mutation-cystine mini-binder. Panel (C) shows mini-binder conjugation with DNA. Panel (D) shows three helix bundle DNA nanostructure. Panel (E) shows molecular representation of mini-binder assemblies on the three-helix bundle DNA. Panel (F) shows molecular surface representation of tri-pdbody bound to the SARS-COV-2 spike ectodomain trimer.

FIG. 19: Minibinder conjugated with DNA and evaluated binding affinity. Panel (A) shows purification of wild type mini-binder and mutant (S78C) mini-binder, Panel (B) shows assessment of the binding affinity of mini-binding by SPR. Panel (C) shows mini-binder conjugation with DNA and purification. Panel (D) shows assessment the binding affinity of mini-binding with DNA by SPR.

FIG. 20: Minibinder binding specificity and avidity of different spike variants. Panel (A) shows schematic representation of competitive assay. Pane (B) shows assessment of the neutralization inhibition assay for minibinder and ACE2, BSA as a negative control. Panel (C) shows assessment the binding affinity of mini-binding with wild type spike and other variants by SPR.

FIG. 21: Different arm tri-pdbodys assembly. Panel (A) shows schematic representation of one, two, and three arm 3 helix bundle. Panel (B) shows characterization of the different arm 3HB by Gel assay. Panel (C) shows imaging of the different arm 3HB by AFM. Panel (D) shows schematic representation of different arm tri-pdbody assembly. Panel (E) shows Characterization of the different arm tri-pdbody by Gel assay. Panel (F) shows imaging of the different arm tri-pdbody by AFM.

FIG. 22: Measurement of binding kinetics of different arm tri-pdbody. Panel (A) shows schematic representation of the binding kinetics of delta spike protein with tri-pdbody. Panel (B) shows equilibrium constraints of the binding to delta spike protein. Panel (C) shows mono, bi, and tri-pdbody response to delta spike protein. Solid lines are global fitting of data to first-order kinetics. Panel (D) shows mono-pdbody response to delta spike protein by SPR. Panel (E) shows bi-pdbody response to delta spike protein by SPR. Panel (F) shows tri-pdbody response to delta spike protein by SPR.

FIG. 23: Measurement of binding kinetics of tri-pdbody and antibody. (A) Schematic of the binding kinetics of delta spike protein with tri-pdbody (B) Tri-pdbody response to wild type spike protein by SPR. (C) Tri-pdbody response to delta spike protein by SPR. (D) Schematic of the binding kinetics of delta spike protein with antibody. (E) antibody response to wild type spike protein by SPR. (E) Antibody response to delta spike protein by SPR.

FIG. 24: Anti-SARS CovID 19 virus infection assay. Panel (A) shows schematic representation of tri-pdbody neutralized the spike protein. Panel (B) shows assessment of the toxicity of tri-pdbody by cell survival assay. Panel (C) shows evaluation of the cell survival after treatment with different SARS-covID19 variants and mixed with tri-pdbody.

FIG. 25: The assembly of mon, bi, and tri-pdbody binding with RBD. Panel (A) shows imaging mono-pdbody binding with RBD protein by AFM. Panel (B) shows Bi-pdbody binding with RBD protein by AFM. Panel (C) shows tri-pdbody binding with RBD protein by AFM.

FIG. 26: Analysis of 3HB-lcb (Synbody) binding to spike protein of mutant strain by agarose gel (EtBr). Panel (A) shows agarose gel analysis of Delta spike incubated with varying molar equivalents of 3HB-lcb (synbody). Using one molar equivalent of the Delta spike results in the complete binding of the synbody. Panel (B) shows agarose gel analysis of Omicron spike incubated with varying molar equivalents of 3HB-lcb (synbody).

FIG. 27: Characterization of SARS-COV-2 pseudovirus. Panel (A) shows NanoSight NTA analysis of the sizes of SARS-COV-2 pseudovirus. Panel (B) shows negative stain EM results of SARS-COV-2 pseudovirus.

FIG. 28: Panel (A) shows microscope images showing GFP expression in Vero-E6-ACE2 or 293T cells 48 hours after incubation with Spike-pseudotyped lentiviral particles with the GFP backbone. For each viral entry protein, 293T and Vero-E6-ACE2 cells were incubated with equal volumes of the virus. Panel (B) shows Immunoblot of overexpressed ACE2 in HEK293T cells and Vero-E6-ACE2 cells.

FIG. 29: Neutralization of wild-type pseudovirus infection by synbody. Inhibition of SARS-COV-2 pseudovirus infection of ACE2-expressing Vero-E6 cells by 10 nM synbody.

FIG. 30: Characterization of synbody-spike protein hybrid complex using negative stain TEM: Panel A) shows TEM image of delta spike protein; Panel B) shows TEM image of spike protein bound with synbody; Panel C) Class 2D of spike protein depicting S1 and S2 subunits; D) Class 2D of synbody (indicated using red arrow) bound with spike protein at S1 subunit.

### DETAILED DESCRIPTION

In a first aspect of the current disclosure, DNA-peptide hybrid molecules are provided. In some embodiments, the DNA-peptide hybrid molecules comprise a DNA nanostructure chemically linked to one or more target-specific binding peptides, wherein the one or more target-specific binding peptides are specific for SARS-COV-2.

In some embodiments, the compositions disclosed herein are used in methods of detecting, purifying, or isolating a target of interest. In some embodiments, the methods comprise contacting a sample containing the target of interest to the DNA-peptide hybrid molecules, wherein the target-specific binding peptide(s) bind one or more sites on the target of interest. In some embodiments, the DNA-peptide hybrid molecules are linked to a solid support or are linked linking to a detectable marker.

In some embodiments, the compositions disclosed herein are used as therapeutics, and are administered to a subject in need thereof to treat or prevent a disease or condition.

### Terminology

Unless defined otherwise, all technical and scientific terms used herein have the same meaning as commonly understood by one of skill in the art to which the invention pertains. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

A range includes each individual member. Thus, for example, a group having 1-3 members refers to groups having 1, 2, or 3 members.

It should also be understood that, unless clearly indicated to the contrary, in any methods claimed herein that include more than one step or act, the order of the steps or acts of the method is not necessarily limited to the order in which the steps or acts of the method are recited.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the term “nanostructure” is a defined structure having at least one dimension (e.g., length, width, thickness) in the nanoscale range (approximately 1 nanometer (nm) to 100 nm).

The term “DNA nanostructure”, as used herein, refers to a nanostructure at least partially composed of DNA assembled in a defined structure and having at least one dimension (e.g., length, width, thickness) in the nanoscale range (approximately 1 nm to 100 nm).

As used herein, “LCB1” refers to a peptide with the sequence DKEWILQKIYEIMRLLDELGHAEASMRVSDLIYEFMKKGDERLLEEAERLLEEVER (SEQ ID NO: 2). More information surrounding the properties and the use of LCB1 can be found in the publication Cao L et al. Science. Vl. 370, No. 6515 pp. 426-431, 2020, incorporated by reference herein in its entirety. In some embodiments, LCB1 is chemically linked to a DNA nanostructure. In some embodiments, more than one LCB1 molecule is chemically linked to one DNA nanostructure.

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, IRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nucleic acid handle”, as used herein, is a nucleic acid attached to or intended for attachment to a polypeptide and having at least some nucleic acid bases available for hybridization to complementary nucleic acid strands of a nucleic acid mold or other structure. Nucleic acid handles may include single-stranded DNA, double-stranded DNA with at least a portion of single-stranded DNA, RNA, aptamers, and peptide nucleic acids (PNAs), or combinations thereof.

The phrase “DNA origami nanostructure” as used herein refers to a nanostructure composed of DNA folded into a precise two- or three-dimensional shape.

As used herein, the term “hyperstable” refers to the unusually high structural stability of a protein or protein nanostructure in terms of its resistance to melting and chemical denaturation. As used herein, a hyperstable protein has a melting temperature of 80° C. or more and is stable in 4M guanidinium chloride.

As used herein, “orthogonal chemical reactions” refers to different chemical reactions that occur selectively and in high yield in the presence of other functional groups. Exemplary orthogonal reactions include, but are not limited to, click chemistry, maleimide chemistry, disulfide formation, oxime formation between an aminooxy group and a ketone/aldehyde, tetrazine/trans-cyclooctene conjugation, enzymatic ligations (e.g., transglutaminase), copper-catalyzed click reactions, and tyrosine oxidation reactions. Various other reactions may include those described in Stephanopoulos, N., “Hybrid Nanostructures from the Self-Assembly of Proteins and DNA”, Chem. 6, pp. 364-405, 2020, incorporated herein by reference.

As used herein, the term “click reaction” refers to the reaction of an azide group with an alkyne group to form a 5-membered heteroatom ring.

As used herein, “DNA-peptide hybrid molecule” refers to a molecule that is comprised of a DNA molecule chemically linked to a peptide molecule thereby generating a DNA-peptide hybrid molecule.

As used herein, “target-specific binding peptide” is a polypeptide molecule that is able to bind to another protein, peptide, or other molecule of interest. Target-specific binding peptides may be chemically linked, for example, to DNA nanostructures or DNA nanocarriers. In some embodiments, more than one target-specific binding peptides are linked to a single DNA nanostructure or nanocarrier. In some embodiments, linking more than one target-specific binding peptides to one DNA nanostructure increases the affinity of the DNA nanostructure-peptide hybrid compared to the target-specific binding protein alone. In some embodiments, the peptide LCB1 is a target-specific binding peptide. As used herein, “target-specific” refers to the property of a molecule having a high affinity for another molecule. In some embodiments, target specific molecules may have a K_{d} or dissociation constant of less than 1 micromolar, or preferably less than 5 nanomolar with a target molecule.

As used herein, “photocleavable linkage” is a chemical link between two or more molecules that can be cleaved upon exposure to light of a given wavelength or energy. In some embodiments, o-nitrobenzyl ester moieties are installed into the DNA backbone of a DNA-peptide hybrid molecule such that, upon exposure to 350 nm ultraviolet (UV) light, the chemical linkages in the DNA molecule are cleaved. In some embodiments, placement of the cleavable linkages is selected such that the cleavage separates the DNA portion of the molecule from the peptide portion of the molecule. Therefore, in the context of a DNA-peptide hybrid molecule, wherein the peptide portion of the molecule binds specifically to a target molecule, the cleavage of the o-nitrobenzyl ester moieties in the DNA portion of the molecule upon exposure to 350 nm UV light effectively separates the target-binding, i.e., peptide portion of the molecule, from the rest of the molecule.

As used herein, “binding affinity” or “affinity” is the strength of the binding interaction between a single molecule and its ligand or binding partner.

As used herein, “binding avidity”, “avidity”, or “functional affinity” is the strength of binding between a molecule comprising multiple target-binding sites and the target molecule. In some embodiments, the DNA-peptide hybrid molecules of the present disclosure comprise multiple target-specific peptides bound to a single DNA nanostructure. Therefore, the avidity of the DNA-peptide hybrid molecule is the strength of the binding of the complete structure of the molecule including the multiple target-specific binding peptides to the target molecule.

As used herein, “immunoglobulin Fc domain” or “Fc domain” refers to the fragment crystallizable domain or the tail region of an antibody that interacts with cell surface receptors called Fc receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. In some embodiments, the DNA-peptide hybrid molecules of the present disclosure comprise an immunoglobulin Fc domain. In some embodiments, the type of Fc domain selected is designed such that the appropriate immune response is instigated by the Fc domain selected. For example, the properties of the Fc domains are known in the art and include the ability to promote antibody directed cellular cytotoxicity (ADCC) and ADCP. As used herein, “antibody directed cellular cytotoxicity” or “ADCC” refers to lysis of target cells coated with antibody by effector cells with cytolytic activity and specific immunoglobulin receptors called Fc receptors, including NK cells, macrophages, and granulocytes.

As used herein, “nanobody” refers to a single monomeric variable antibody domain, also known as single-domain antibodies (sdAbs) that are able to bind selectively to a specific antigen.

As used herein, “antigen” refers to a molecule that is capable of stimulating the immune system of a subject.

As used herein, “paratope” refers to region of an antibody that binds to the antigen-binding site (epitope) of the target molecule.

In some embodiments, the DNA-peptide hybrid molecules of the present disclosure which, in some embodiments, are designed to bind to a target molecule, can be “sized” or “tuned” to match the distance and/or arrangement of the binding domains in the target molecule. Put another way, if, for example, the target molecule contains two target-binding domains for which the DNA-hybrid molecule is designed to bind, that are 5 nm apart, the DNA nanostructure may be sized or tuned such that the target-specific binding peptides, when attached to the DNA nanostructure, are located about 5 nm apart in a conformation that enables favorable access of the target-specific binding peptides to the target-binding domains. Thus, without being limited by any theory or mechanism, this tunable property of the compositions of the current disclosure is thought to enable rational design of DNA nanostructures that takes advantage of the property of avidity of multiple binding domains binding to a single target molecule. In essence, being able to be tuned increases the functional affinity of the DNA-hybrid molecule to its target molecule when compared to the affinity of a similar molecule that does not present the target-specific binding peptides in a conformation that allows them to be accessible to the target binding regions of the target molecule.

In a second aspect of the current disclosure, methods of treatment are provided. In some embodiments, the method comprises administering a DNA-peptide hybrid molecule comprising a DNA nanostructure chemically linked to one or more target-specific binding peptides, wherein the one or more target-specific binding peptides are specific for a molecule associated with a disease or disorder in an amount sufficient to treat the disease or disorder.

As used herein, “infectious disease” refers to diseases caused by pathogenic microorganisms including, for example, bacteria, fungi, viruses and eukaryotic parasites. In some embodiments, the infectious disease is coronavirus disease discovered in 2019 (COVID-19) caused by severe acute respiratory syndrome coronavirus 2 (SARS-COV-2).

As used herein, “autoimmune disease” refers to a disease or disorder wherein a subject's immune system attacks normal cells and tissues in the subject.

As used herein, “cancer” refers to a large group of cell proliferative disorders caused by an uncontrolled division of abnormal cells.

As used herein, “psychiatric disease or disorder” refers to wide variety of behavioral or mental patterns that cause significant distress or impairment of personal functioning in affected subjects. Psychiatric diseases or disorders are caused by abnormal functioning of the central nervous system.

As used herein, “environmental exposure” refers to contact with chemical, biological, or physical substances found in air, water, food, or soil that may have a harmful effect on a person's health.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with the target molecule to which the disclosed compositions are targeted.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Sequence of SARS-COV-2 surface glycoprotein also known as “spike” protein (SEQ ID NO: 1):

| | |
|---|---|
| MFVFLVLLPL VSSQCVNLTT RTQLPPAYTN SFTRGVYYPD KVFRSSVLHS TQDLFLPFFS | 60 |
| NVTWFHAIHV SGTNGTKRFD NPVLPFNDGV YFASTEKSNI IRGWIFGTTL DSKTQSLLIV | 120 |
| NNATNVVIKV CEFQFCNDPF LGVYYHKNNK SWMESEFRVY SSANNCTFEY VSQPFLMDLE | 180 |
| GKQGNFKNLR EFVFKNIDGY FKIYSKHTPI NLVRDLPQGF SALEPLVDLP IGINITRFQT | 240 |
| LLALHRSYLT PGDSSSGWTA GAAAYYVGYL QPRTFLLKYN ENGTITDAVD CALDPLSETK | 300 |
| CTLKSFTVEK GIYQTSNFRV QPTESIVRFP NITNLCPFGE VFNATRFASV YAWNRKRISN | 360 |
| CVADYSVLYN SASFSTFKCY GVSPTKLNDL CFTNVYADSF VIRGDEVRQI APGQTGKIAD | 420 |
| YNYKLPDDFT GCVIAWNSNN LDSKVGGNYN YLYRLFRKSN LKPFERDIST EIYQAGSTPC | 480 |
| NGVEGFNCYF PLQSYGFQPT NGVGYQPYRV VVLSFELLHA PATVCGPKKS TNLVKNKCVN | 540 |
| FNFNGLTGTG VLTESNKKEL PFQQFGRDIA DTTDAVRDPQ TLEILDITPC SFGGVSVITP | 600 |
| GTNTSNQVAV LYQDVNCTEV PVAIHADQLT PTWRVYSTGS NVFQTRAGCL IGAEHVNNSY | 660 |
| ECDIPIGAGI CASYQTQTNS PRRARSVASQ SIIAYTMSLG AENSVAYSNN SIAIPTNFTI | 720 |
| SVTTEILPVS MTKTSVDCTM YICGDSTECS NLLLQYGSFC TQLNRALTGI AVEQDKNTQE | 780 |
| VFAQVKQIYK TPPIKDFGGF NFSQILPDPS KPSKRSFIED LLFNKVTLAD AGFIKQYGDC | 840 |
| LGDIAARDLI CAQKFNGLTV LPPLLTDEMI AQYTSALLAG TITSGWTFGA GAALQIPFAM | 900 |
| QMAYRFNGIG VTQNVLYENQ KLIANQFNSA IGKIQDSLSS TASALGKLQD VVNQNAQALN | 960 |
| TLVKQLSSNF GAISSVLNDI LSRLDKVEAE VQIDRLITGR LQSLQTYVTQ QLIRAAEIRA | 1020 |
| SANLAATKMS ECVLGQSKRV DFCGKGYHLM SFPQSAPHGV VFLHVTYVPA QEKNFTTAPA | 1080 |
| ICHDGKAHFP REGVFVSNGT HWFVTQRNFY EPQIITTDNT FVSGNCDVVI GIVNNTVYDP | 1140 |
| LQPELDSFKE ELDKYFKNHT SPDVDLGDIS GINASVVNIQ KEIDRLNEVA KNLNESLIDL | 1200 |
| QELGKYEQYI KWPWYIWLGF IAGLIAIVMV TIMLCCMTSC CSCLKGCCSC GSCCKFDEDD | 1260 |
| SEPVLKGVKL HYT | |

The “alpha” variant of SARS-COV-2, or B.1.1.7 variant has the following mutations: 69-70del, N501Y, and P681H.

The “beta” variant of SARS-COV-2, or B.1.351 variant has the following mutations: K417N, E484K and N501Y.

The “gamma” variant of SARS-COV-2, or P.1 variant has the following mutations: K417T, E484K, and N501Y.

The “delta” variant of SARS-COV-2, or B.1.617.2 variant has the following mutations: L451R, T478K, and P681R.

While four variants are described above, additional variants, as they develop, are identified and are sequenced, may be used in the context of the present invention. Based on the present disclosure, the skilled artisan would understand how to make and use such variants.

### EXEMPLARY EMBODIMENTS

1. A DNA-peptide hybrid molecule comprising a DNA nanostructure chemically linked to one or more target-specific binding peptides, wherein the one or more target-specific binding peptides are specific for SARS-COV-2.
2. The composition of embodiment 1, wherein the DNA nanostructure is selected from the group consisting of: a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool.
3. The composition of embodiment 2, wherein the DNA nanostructure comprises one of a three-helix bundle or a four-helix bundle.
4. The composition any of the previous embodiments, wherein the target-specific binding peptide is LCB1.
5. The composition of any of the preceding embodiments, wherein the distance between the target-specific binding peptides on the fully assembled DNA-peptide hybrid molecule is about the distance between the sites where the target-specific binding peptide binds to the native SARS-COV-2 homotrimeric spike protein.
6. The composition of any of the preceding embodiments, wherein the DNA-peptide hybrid molecule has greater affinity for SARS-COV-2 spike protein compared to the one or more target-specific binding peptides alone.
7. The composition of any of the preceding embodiments, wherein the DNA-peptide hybrid molecule has a K_{d} of less than about 5 nM for each of the spike protein of SARS-COV-2 alpha, beta, gamma, and delta variants.
8. The composition of any of the preceding embodiments, wherein the synthetic DNA-peptide hybrid molecule further comprises an immunoglobulin Fc domain.
9. The composition of embodiment 8, wherein binding of the DNA-peptide hybrid molecule to SARS-COV-2 spike protein induces immune cells to engage in antibody dependent cellular cytotoxicity (ADCC).
10. The composition of any of the preceding embodiments, wherein the one or more target-specific binding peptides and the DNA nanostructure are linked through a disulfide bond or through copper-free click chemistry between a protein bearing the non-canonical amino acid 4-azidophenylalanine and DNA linked to cyclooctyne.
11. The composition of any of the preceding embodiments, wherein the chemical linkage of the one or more target-specific binding peptides to the DNA nanostructure is cleavable.
12. The composition of embodiment 11, wherein the chemical linkage comprises a photocleavable linkage.
13. A method of treating a subject in need thereof, the method comprising administering to the subject a DNA-peptide hybrid molecule comprising a DNA nanostructure chemically linked to one or more target-specific binding peptides, wherein the one or more target-specific binding peptides are specific for SARS-COV-2, in an amount sufficient to treat SARS-CoV-2 infection.
14. The method of embodiments 13, wherein the DNA nanostructure is selected from the group consisting of: a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool.
15. The method of any of embodiments 13 or 14, wherein the DNA nanostructure comprises one of a three-helix bundle or a four-helix bundle.
16. The method of any of embodiments 13-15, wherein the one or more target-specific binding peptides is LCB1.
17. The method of any of embodiments 13-16, wherein the distance between the target-specific binding peptides on the fully assembled DNA-peptide hybrid molecule is about the distance between the sites where the target-specific binding peptide binds to the native SARS-CoV-2 homotrimeric spike protein.
18. The method of any of embodiments 13-17, wherein the DNA-peptide hybrid molecule has greater affinity for SARS-COV-2 spike protein than the target-specific peptide alone.
19. The method of any of embodiments 13-18, wherein the DNA-peptide hybrid molecule has a K_{d} of less than about 5 nM for each of the spike protein of SARS-COV-2 alpha, beta, gamma, and delta variants.
20. The method of any of embodiments 13-19, wherein the DNA-peptide hybrid molecule further comprises an immunoglobulin Fc domain.
21. The method of embodiment 20, wherein binding of the DNA-peptide hybrid molecule to SARS-COV-2 spike protein induces immune cells to engage in antibody dependent cellular cytotoxicity (ADCC).
22. The method of any of embodiments 13-21, wherein the one or more target-specific binding peptides and the DNA nanostructure are linked through a disulfide bond or through copper-free click chemistry between a peptide bearing the non-canonical amino acid 4-azidophenylalanine and DNA linked to cyclooctyne.
23. The method of any of embodiments 13-22, wherein the chemical linkage of the one or more target-specific binding peptides to the DNA nanostructure is cleavable.
24. The method of embodiment 23, wherein the chemical linkage comprises a photocleavable linkage.
25. The method of any of embodiments 13-24, wherein the subject in need thereof is infected with SARS-COV-2.
26. The method of any of embodiments 13-24, wherein the subject in need thereof is not infected with SARS-COV-2.
27. The method of any of embodiments 13-26, wherein the DNA-peptide hybrid molecule is administered as a pharmaceutical composition comprising one or more suitable excipients, solvents, or vehicles whereby the pharmaceutical composition can be effectively administered to a subject in need thereof.

### EXAMPLES

### Example 1

### Blocking Protein-Protein Interactions is Crucial for Biological Studies.

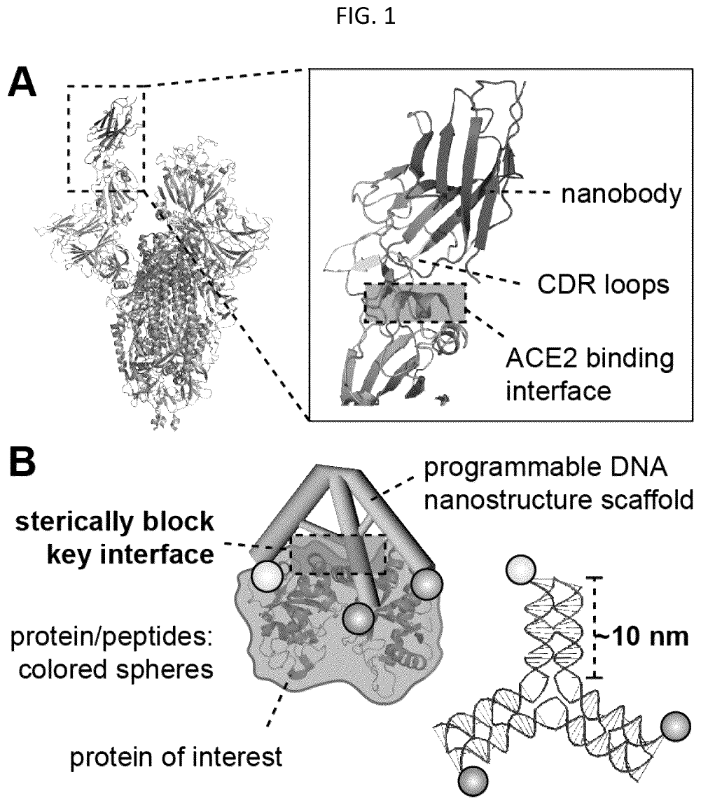

The ability to block protein-protein interactions (PPIs) is crucial not just for therapeutic purposes—e.g. neutralizing antibodies for pathogenic threats like SARS-COV-2, or small molecule drugs for cancer therapy—but also for fundamental biological studies. Countless biological processes are mediated by protein-protein interactions, such as cell-cell interactions, signal transduction, cell-matrix interactions, immune system recognition, and many others, but it can be difficult to block these interactions with high affinity and specificity. Approaches like small molecule drugs, or peptides found through rational design or high-throughput evolutionary methods like phage, mRNA, or ribosome display are often hindered by lack of binding to the key protein-protein interface. Antibodies can block PPIs, but again must target a key interface (FIG. 1A). Furthermore, these methods are generally not reversible or triggerable, and cannot be switched “on” and “off” on-demand with simple triggers. Creating a nanostructure that can switch PPIs on in a stimulus-responsive fashion (especially using light) would enable basic biology studies in targets that are not amendable to traditional optogenetic approaches. Furthermore, PPIs can span a large range of sizes, and it can be especially difficult to block multivalent interactions, as in viruses. Disclosed herein is a protein-DNA nanostructure platform whose dimensions can be precisely tuned to “match” a protein target, enveloping it and blocking its function (FIG. 1B). This approach does not rely on selectively binding the key interface; rather, inventors use a DNA nanoscaffold to position multiple peptides or proteins in 3D space to bind to different patches of the protein target, with the remainder of the structure sterically occluding the binding interface. The ability to reverse the nanostructure assembly (using precise stimuli like light) will impart spatiotemporal control to blocking the interaction.

### Multivalent Binding Enhances Affinity and Expands Target Scope.

One way to dramatically increase affinity for a target is by leveraging avidity: positioning multiple binding groups so that they can act cooperatively. Antibodies like IgG and IgM are intrinsically multivalent, although their geometry cannot be tuned to match the target. Extensive work in bionanotechnology has sought to rationally design multivalent binding agents for biomaterial applications. Most of these examples simply rely on a high density of the binding agents for activity, but a number of recent efforts have focused on matching the target size and valency with greater precision. For example, intrinsically symmetric assemblies can be targeted with designed homo-oligomeric binding agents. In one report from 2017, Strauch el al. reported a de novo designed homotrimeric protein grafted with a complementarity determining region (CDR) loop derived from a hemagglutinin (HA)-binding antibody could neutralize influenza with an IC₅₀ in the picomolar range. Precisely matching the HA trimer geometry (namely the distance between monomers and the threefold rotational symmetry) was critical to the binding affinity. Using a similar design principle, in 2019 Kwon et al. reported a starshaped DNA nanostructure that positioned aptamers to match the distance and fivefold symmetry of the dengue virus coat proteins, resulting in potent binding and virus inhibition (EC₅₀=2 nM). Once again, the ability to recapitulate the geometry of the capsid proteins with the aptamer was critical; structures that were too large or too small, or that had fewer or more than five ligands, did not bind as effectively. Furthermore, this example demonstrated the great potential of DNA as a programmable scaffold with controllable dimensions, relying on the precise valence and distances of the star-shaped scaffold to recapitulate the capsid protein symmetry.

The above examples, however, are restricted to homo-oligomeric targets like HA or viral capsids. Extending this paradigm to multiple different targets (either on a single protein or a protein complex) would dramatically expand the range of possible targets. Even simple DNA duplexes can be used as “molecular rulers” to position two binding groups, such as peptides or scFv molecules with a tunable distance to bind two separate sites on a target and enhance binding. In the latter example, two scFv fragments (either identical or different) could target HIV-1 virion spike proteins and improve virus neutralization by over 100-fold, whereas native IgG molecules were too large to effectively bind. Recent work from Zhou et al. also demonstrated that a DNA tile bearing two aptamer loops could be evolved to target non-overlapping sites of a target protein with femtomolar affinity, with the tile imparting the appropriate spacing to match the protein size. In the present disclosure, the inventors demonstrate that a DNA nanostructure can be designed to position multiple protein binding groups with precise spatial control, but without the scaffold size limitations of antibodies or antibody mimetics. In some embodiments, the method and structures can position multiple (2-3) protein/peptide-based ligands, on a size- and shape-programmable scaffold. In some embodiments, these nanoscale synthetic antibodies, hereinafter “DNA-peptide hybrid molecules,” will be designed and optimized/“evolved” in silico using coarse-grained molecular dynamics simulations, in a feedback loop with experimental results.

### DNA Nano-Scaffolds Possess Several Key Advantages Over Other Display Methods.

The use of DNA nanostructures—such as DNA origami, multi-helical bundles, branched tiles, wireframe cages, or single stranded “brick” assemblies—to display peptides or proteins in a multivalent fashion has certain key benefits over other scaffolds like proteins, polymers, or self-assembled nanoparticles/fibers. These advantages include: (1) Facile presentation of multiple polypeptides (either identical or different) with stoichiometric control and user-defined valency; (2) Control over the spacing of the peptides with ˜3-5 nm resolution; (3) User-defined size and shape of the ultimate structure to best match a target size (up to tens of nanometers); (4) Attachment of the final targeting assembly with other nanoscale carriers like liposomes or nanoparticles via DNA hybridization; (5) Potential for multivalent, or bi-/multi-specific structures by oligomerizing individual DNA-peptide hybrid molecules using DNA; (6) Steric blockage of protein-protein interactions due to their large size; (7) Demonstrated stability and functionality in vivo of either bare nanostructures or after stabilization using simple peptide coatings; (8) Large scale (˜$100/gram) production using recent breakthrough DNA production methods; (9) Dynamic assembly/disassembly of structures using light or input displacement strands. (10) Ability to be shielded from the immune system, or to stimulate an immune response depending on the desired application. (11) Capacity for intracellular delivery and subcellular trafficking. (12) Potential to target assembled protein complexes by combining binders to distinct components of the complex on a DNA scaffold. Inventors also highlight that using a rigid DNA nanostructure (as opposed to a simple dsDNA molecular ruler) will enable enhanced binding due to lower entropic penalties, and the use of three or more binding peptides/proteins with precise display in 3D space.

One aspect of the disclosed technology is to use a DNA nano-scaffold to control the spatial orientation of multiple binding peptides or proteins, to create a highly specific synthetic blocking agent for protein-protein interactions. In antibodies, a large portion of the sequence is dedicated to positioning a few key CDR loops in the correct conformation; in the inventor's work, inventors effectively decouple this structural component from the binding agents. Unlike antibodies, however, inventors' structures will be designed to match the given target size and geometry. This will enable not only tighter binding (even if the individual peptides/proteins have only modest affinity), but also blocking of the target cell surface receptors due to the steric bulk provided by the scaffolding nanostructure. Crucially, this method enables peptides that bind to areas away from the targeted interface to be converted to a blocking function through the appended nanoscaffold. Because our approach can use both short, synthetic peptides and larger, folded proteins, it serves as a rapid way to quickly extend binding agents found from other approaches (e.g. phage/mRNA/yeast/ribosome display, de novo designed proteins, or novel nanobodies or scFv fragments) to multivalent scaffolds. In addition to using reported peptide/proteins and designing nanostructures to best bind a target, inventors will also find novel binding agents for fibrin/fibrinogen, and attach them to a DNA scaffold in a multivalent fashion. All of these approaches require seamless molecular integration of the protein/peptide groups with a DNA nanoscaffold, with control over the linker length and rigidity, so tailored protein-DNA bioconjugation will play a critical role in these studies.

Another aspect of the disclosed technology is the in silico screening and optimization of hybrid peptide/protein-DNA nanostructures. Considerations in designing a hybrid peptide-DNA nanostructure include balancing competing demands: (1) enough rigidity so that there is no entropic penalty to binding, yet (2) sufficient flexibility to tolerate thermal fluctuations and imperfections in the design. To tune these competing forces, inventors develop the first integrated, coarse-grained model of protein-DNA nanostructures, where both molecules can be parameterized in a way that is accurate and computationally tractable. The model will in turn allow us to computationally screen multiple different DNA nanostructure designs, both in terms of geometry and strategic introduction of flexible/bulged sections, and to test the effect of peptide-DNA linker length and flexibility. Inventors employ computational models to best estimate pairwise distances between two binding agents whose binding site is unknown, and then use these distances as guidelines to design high-affinity blocking agents.

Currently, the major obstacle of in silico design in therapeutics are the system sizes and timescales involved in studying the binding pathways, as well as the correct parametrization of the models that predict binding interactions. As DNA nanostructures contain hundreds to several thousands of nucleotides, they are not amenable to atomistic-resolution computational studies that would sample their binding pathways to proteins. However, the coarse-grained approach allows for efficient sampling, making in silico evolutionary design possible by automatically generating and testing in simulation the binding of libraries of DNA nanostructures. Thus, this work will develop a new efficient design framework for automated evolutionary design, analysis and optimization of peptide/protein-DNA nanoscaffolds. Such a platform can greatly reduce experimental costs and speed-up development of high-affinity blocking DNA-peptide hybrid molecules. Although our work will develop and validate the system on the SARS-COV-2 spike protein and fibrinogen as model systems, the emphasis will be on a workflow that can be readily adapted to new targets and new binding agents.

### Overview:

Disclosed herein are methods for designing DNA nanostructures that can spatially display 2-3 binding ligands (primarily peptides and proteins, though aptamers can also be employed) that bind to different portions of a given protein target. In some embodiments, the methods include accurate methods for computationally modeling the hybrid protein/peptide-DNA nanostructure, and “docking” it with the target without too great of an entropic cost. Described herein, therefore, is an integrated computational-experimental pipeline, where coarse-grained simulation methods will be used to design an initial set of DNA-peptide hybrid molecules that can be experimentally tested for binding. The results of these experiments will be used to refine the models and generate a library in silico of slightly mutated nanostructures, the best-performing of which will be selected for future rounds of experimental characterization.

In a first aspect, a target for which multiple binding groups are known—the SARS-CoV-2 spike protein receptor binding domain (RBD)—will be used as a test bed in order to develop and benchmark the method. The inventors will develop DNA-peptide hybrid molecules with three identical binding groups that target the known ACE2 binding site of the RBD. Inventors will then use one of these binding agents in conjunction with recently reported molecules that bind to a different region of the spike protein to develop hetero-bivalent structures. This process will involve novel chemical strategies for integrating the proteins/peptides with the DNA scaffold, optimizing the computational methods used, and testing DNA-peptide hybrid molecule “activity” by blocking the RBD interaction with the ACE2 receptor in a reversible fashion. In a second aspect, inventors use phage display to find several new nanobodies for fibrinogen, and then use these to discover heterobi- and tri-valent DNA-peptide hybrid molecules that bind to this target and block its activity in a stimulus-responsive, light-switchable fashion.

Develop DNA-Peptide Hybrid Molecules for Blocking the SARS-COV-2 Spike Protein. Rationale:

The COVID-19 pandemic has highlighted the need for high-affinity binding/blocking agents for viral threats like SARS-COV-2. As a result, there are a number of promising protein and peptide ligands for the spike trimer receptor binding domain (RBD), which is presented as a homotrimer with a known crystal structure on the capsid surface. Designing a DNA-peptide hybrid molecule that positions three identical proteins/peptides with a geometry and distances that match the RBD trimer will serve as an ideal test bed for both DNA nanostructure synthesis, but also to validate and optimize the theoretical model and computational pipeline. By the end of this aspect, inventors have demonstrated that a DNA scaffold bearing three identical protein/peptide binding groups can serve as a high-affinity blocking agent for a virus.

Synthesize RBD-Binding Proteins and Peptides and Conjugate them to DNA.

Several protein/peptides have been reported that target the SARS-COV-2 spike protein RBD and can neutralize virus association with the target ACE2 receptor. In particular, inventors explore three categories of such binders: (1) a de novo designed mini-binder proteins reported by Cao et al. and Linsky et al. that target RBD with IC50 values ranging from femtomolar to nanomolar; (2) several nanobodies that bind with nanomolar or better affinity; (3) short synthetic peptides that are highly tractable but tend to bind more weakly than proteins. Inventors highlight that one of the nanobodies inventors will investigate was trimerized using a Gly-Ser linker and achieved femtomolar binding affinity and picomolar virus inhibition, despite using a flexible linkage and linear concatenation via genetic fusion. Thus, our nanostructure-scaffolded, size/geometry-matched approach may give even greater affinity by reducing the entropic penalties for rearrangement to the correct geometry.

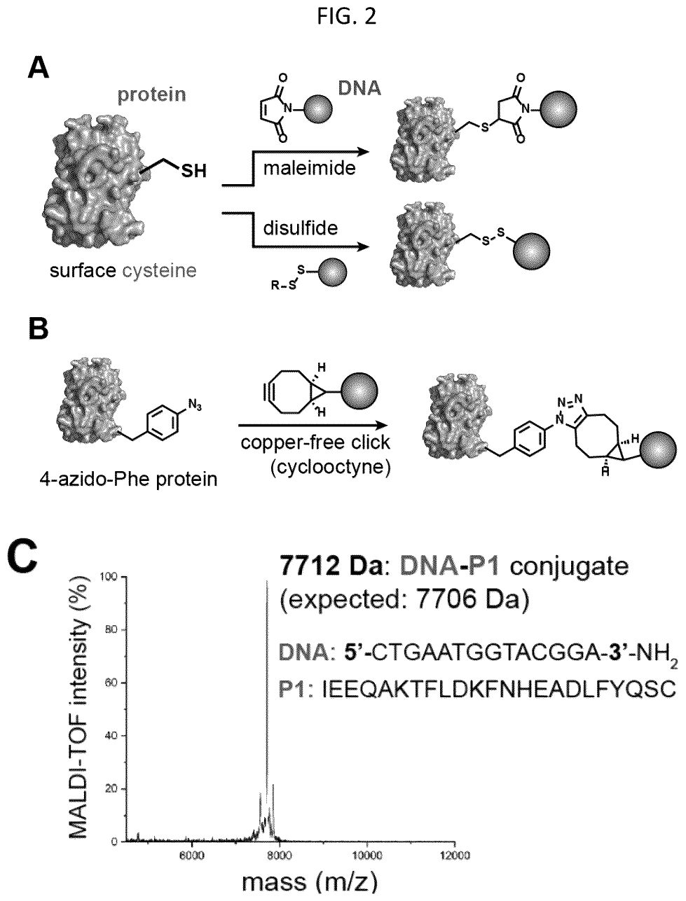

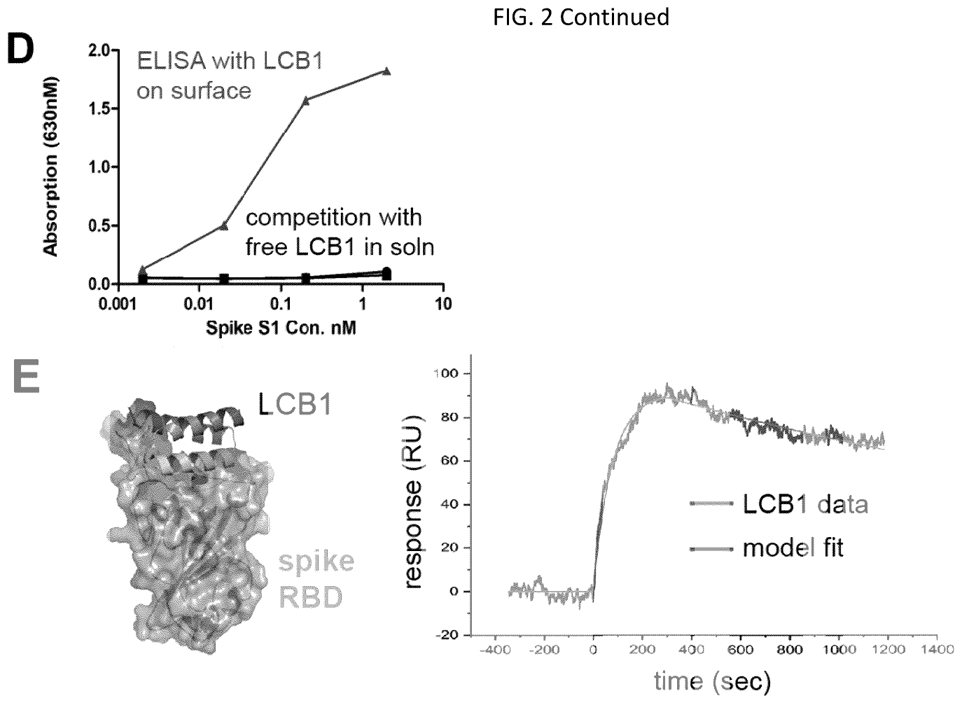

All polypeptides will be conjugated to DNA one of two ways, both of which have been extensively used in PI Stephanopoulos's lab: (1) via a unique, mutagenically-introduced cysteine using a bifunctional linker (FIG. 2A); and (2) copper-free click between a protein bearing the noncanonical amino acid 4-azidophenylalanine and cyclooctyne-DNA (FIG. 2B). Proteins will be expressed recombinantly in E. coli, and peptides will be synthesized on solid phase using standard Fmoc-protected amino acids. Conjugates will be purified using anion exchange or reverse phase chromatography, and characterized via polyacrylamide gel electrophoresis and MALDI-TOF mass spectrometry. The selected binding groups have a range of affinities (from picomolar to low micromolar), which will allow us to determine the range of affinity enhancements imparted by the multivalent scaffold. Recent experiments creating nanobody heterodimers using flexible amino acid linkers have shown affinity enhancements of 4-22 fold, so inventors expect constructs to be at least within this range, with potentially much higher affinities due to the better-defined 3D presentation of the ligands. Preliminary data: The LCB1 protein reported by Cao et al. via recombinant expression followed by nickel affinity and anion exchange chromatography methods has been successfully expressed. A unique mutagenic cysteine has been incorporated into this protein and coupled to DNA using a bifunctional linker. In addition, synthesis of synthetic peptides (up to 50 amino acids) with noncanonical azide residues for copper-free click coupling to DNA (FIG. 2C), as well as nanobody proteins for creating hybrid protein-DNA nanomaterials has been routinely performed.

### Test RBD Binding Activity of Peptides/Proteins and DNA Conjugates.

To test the ability of the synthesized peptides/proteins to bind to the SARS-COV-2 RBD, inventors employ two methods: (1) an ELISA assay using the RBD and its targeting antibody; and (2) surface plasmon resonance (SPR), which was used in the characterization of most of the binding groups mentioned above, and enables greater insight into on- and off-rates of the binding molecules. Preliminary data: Inventors have probed the binding of our in-house expressed LCB1 to RBD using both ELISA and an SPR assay. The LCB1 protein was adsorbed to the surface, followed by exposure to varying concentrations of the monomeric spike RBD protein; the amount of RBD adhered was then probed with a primary antibody and a secondary antibody-HRP conjugate. The RBD protein did indeed bind to the LCB1, with a K_{d} in the 100-200 pM range, similar to reported values (FIG. 2D, red curve). The binding could also be abolished by competition with free LCB1 in solution (FIG. 2D, black curve), further confirming that the RBD was not nonspecifically adsorbing to the surface. The binding was also be probed by SPR (FIG. 2E) and demonstrated a K_{d}˜9 nM, consistent with reported results.

Inventors test the LCB1-DNA conjugate—and all the peptide/protein-DNA hybrids made in an analogous fashion—in the same manner, cognizant of the fact that the DNA handle could decrease the binding affinity. Although the attachment site for DNA has been engineered to be distant from the RBD-binding interface, it may be necessary to screen several attachment sites, as well as linker identities (e.g. alkyl, aryl, PEG) and lengths. Mutated peptide/protein molecules, where the binding interface residues are scrambled to abolish binding, will be used as controls. Inventors will use SPR to determine rates of binding (kon, koff), and thus the Kd values. All conjugates will be compared with the original (i.e. non-DNA-conjugated) binding groups as positive controls.

### Design, Synthesize, and Characterize Hetero-Trivalent Peptide/Protein-DNA Nanostructures.

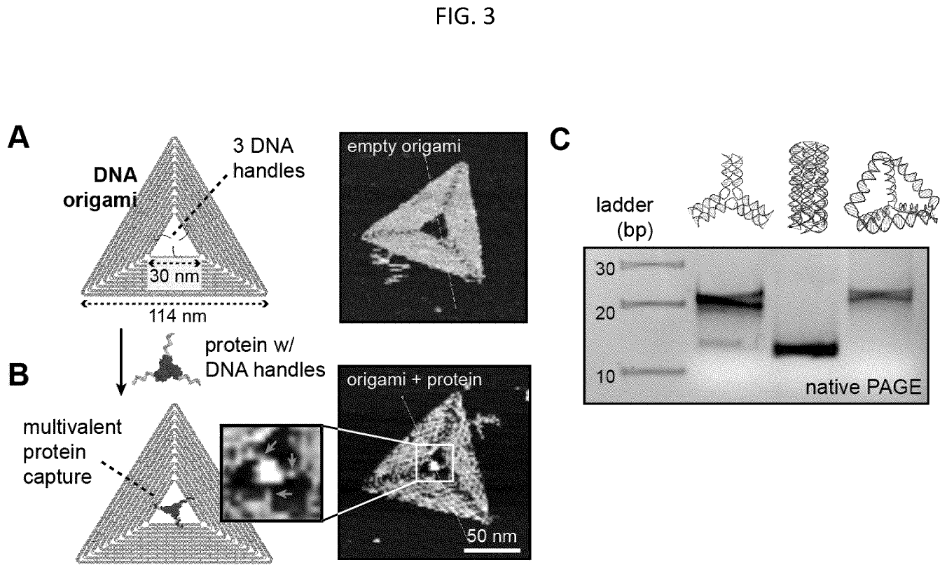

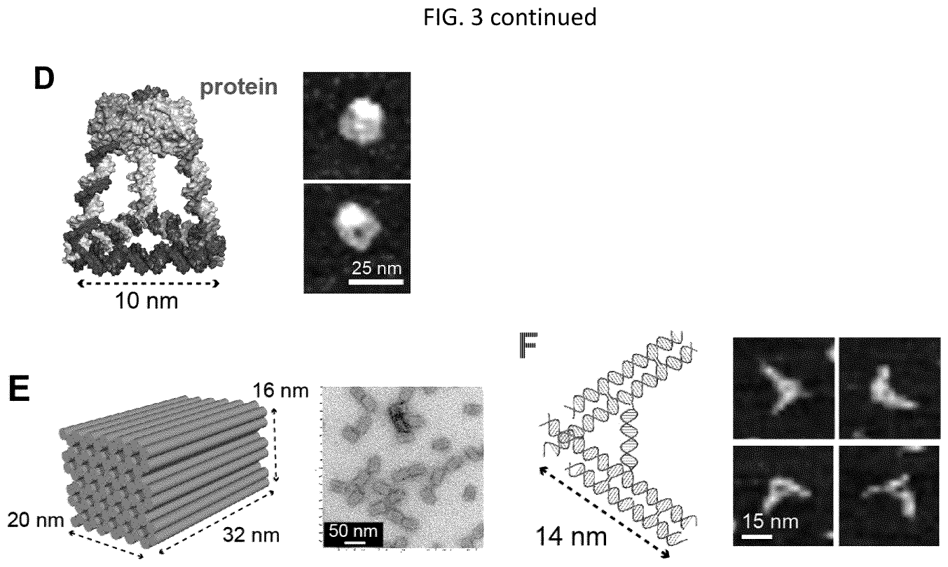

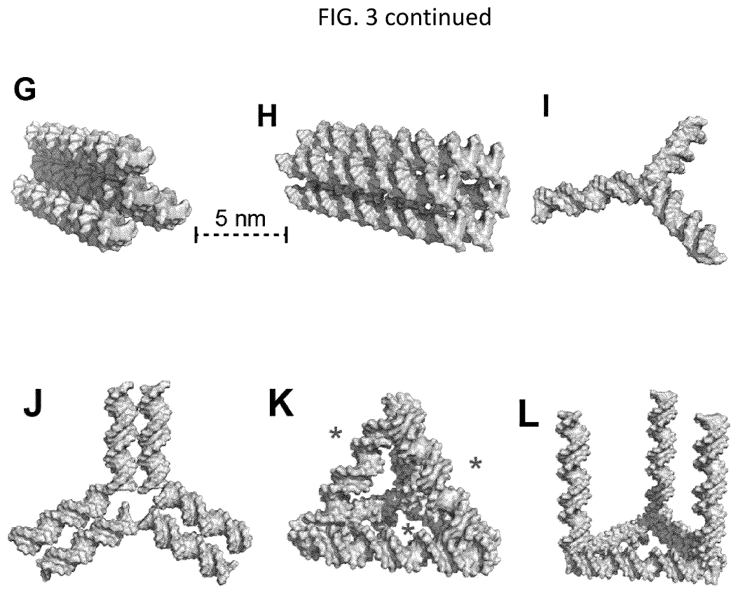

The ideal DNA nano-scaffold for hetero-trivalent presentation of the above peptide/protein-DNA conjugates is a structure that is reasonably rigid (to avoid entropic penalties in nanostructure reconfiguration), and roughly size-matched to the spike RBD trimer diameter (˜7-8 nm). Nanostructures scaffolds will be assembled using thermal annealing of the constituent strands, and purified using either spin filtration, gel excision, or anion exchange chromatography. Single-stranded DNA handles will be included for attachment of peptide/protein-DNA conjugates, and successful incorporation will be probed using gel shift assays and/or using fluorescently tagged peptides/proteins. Nanostructures with zero, one, and two handles for peptide/protein incorporation will be synthesized to probe the effect of not just binding group, but also valency; indeed, this straightforward tunability is an advantage of DNA nanoscaffolds. Preliminary data: A series of DNA nanostructures were designed and used, including (FIGS. 3A-F): triangular DNA origami structures; tetrahedral wireframe cages, six-helix bundles, block-like origami cuboids, reconfigurable tweezers, double crossover tiles, and branched three-way junctions, among many others. These examples include both all-DNA nanostructures, as well as structures that precisely integrate proteins in a multivalent fashion (FIGS. 3B,D) The lab has extensive experience with DNA design software (e.g. Cadnano, Tiamat) as well as techniques and access to facilities to analyze the nanostructures (gel electrophoresis, AFM, TEM).

For this work, inventors will primarily focus on simpler DNA nanostructures (rather than full-size origami) in order to better match the protein size, and to improve the overall scalability of the final assemblies. Towards this end, inventors test structures like four- and six-helix bundles (FIGS. 3G,H), which are cylindrical objects ˜5-7 nm in diameter and highly rigid due to multiple crossover strands linking them together. Inventors will also test three-way tile junctions, tetrahedral cages, and other wireframe assemblies (FIG. 3I-L) that vary in flexibility and shape. These shapes can be tuned over a range of sizes (5-20 nm, though larger structures are also possible with more complex designs), and the valency can be varied from 2-4 ligands readily. They each contain multiple sites for attachment of proteins/peptides; the simplest is at the end of helices by extending the structural strands with ssDNA handles, but the ligands can also be coupled directly to the structural strands and displayed at arbitrary locations along a helix (i.e. not just the ends) by introducing a nick point, or directly conjugated to the backbone of any constituent helix with single-nucleotide precision.

### Develop a Computational Model for Simulating Hybrid Peptide/Protein-DNA Nanostructures.

One bottleneck to developing the proposed DNA-peptide hybrid molecules is that no model exists for the design of hybrid polypeptide-DNA nanostructures (unlike for proteins where packages like Rosetta50 exist for modeling structure and designing novel binding groups). Accurately representing the 3D spatial display of multiple heterogeneous molecules on a DNA nanostructure would allow more accurate matching of the hybrid structure to the target. Such a model would also enable the in silico “mutagenesis” and screening of designs that best match the binding sites on the target in order to guide experimental realization. Preliminary data: The oxDNA tool, a coarse-grained model of DNA that reproduces mechanical, structural and thermodynamic properties of both single-stranded (ss) and double-stranded (ds) DNA will be used. The model has been used in a range of settings, from biophysical studies of DNA to probing the assembly of nanostructures and active nanodevices, usually with good agreement with existing experimental data. OxDNA can efficiently simulate nanostructures consisting of up to tens of thousands of nucleotides and captures timescales that correspond to tens of milliseconds in experiment 51. Recently, an extension of the model was introduced: ANM-oxDNA, that uses the oxDNA model for DNA and also represents protein structures and short peptides using the anisotropic-network-model (ANM) to capture their basic dynamics and conformations. The model is able to reproduce the structure of protein-DNA hybrid structures previously realized in Stephanopoulos lab. Currently, the model does not predict de novo interactions between peptides and proteins, and the possible interactions have to be explicitly specified based on prior knowledge of the binding sites. The model can, however, very quickly sample nanostructure diffusion well as its binding trajectory to a protein. Our prior analysis has shown that the simulation can efficiently sample the possible conformations of a DNA nanostructure- and the regions that a multivalent binder can cover on a protein-within less than 1 GPU-hour for a protein and a nanostructure system consisting of several hundred residues in total.

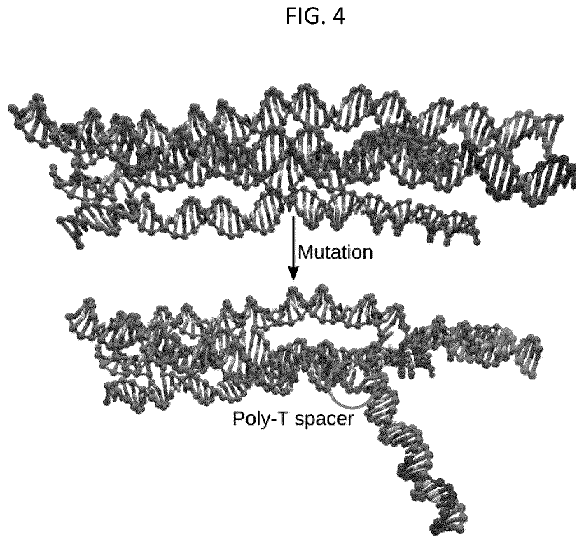

Here, inventors implement an automated in-silico nanostructure mutation generation using our recently developed ox View design tool for nucleic acid nanotechnology, which was recently extended to also support protein structure representation. The initial design for a multivalent peptide/protein-DNA nanostructure can be either imported from other DNA nanotechnology design tools or created directly in oxView. Inventors will then implement an automated algorithm for introducing “mutations” to the structure design, which will include: changing the position for peptide/protein attachment, extending/shortening dsDNA and ssDNA segments in the nanostructure, and introducing bulges and junctions into the design (FIG. 4). Inventors will further implement a docking protocol that calculates the entropy difference between the bound and unbound structure, and enthalpy that is based on provided scoring function that can be imported from peptide-protein docking tools.

Use the Experimental and Computational Pipeline to Optimize DNA-Peptide Hybrid Molecule Structures.

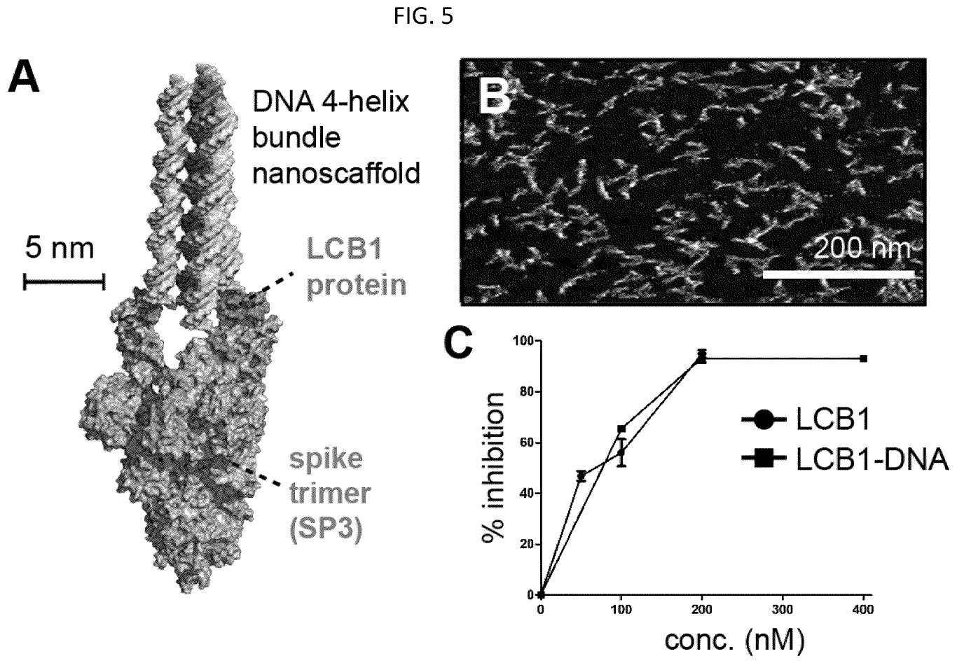

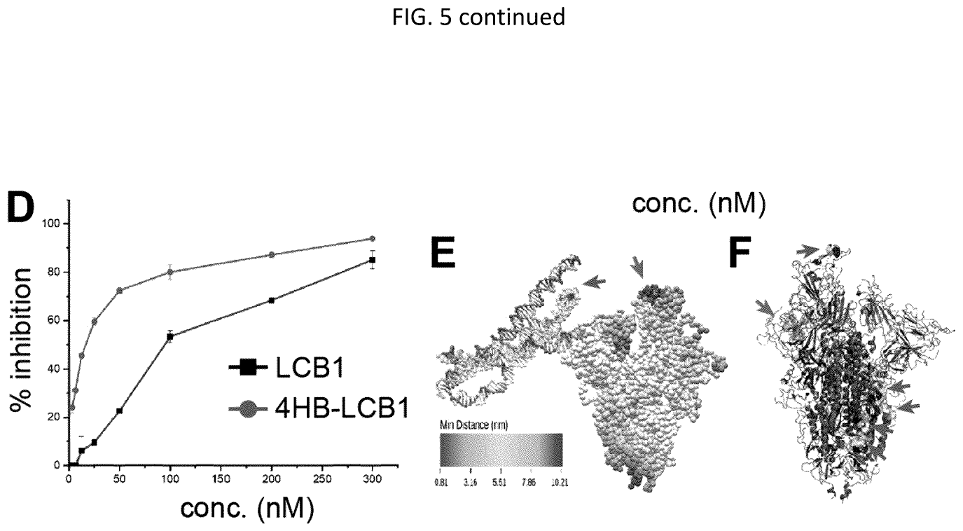

Following synthesis of hetero-trivalent DNA-peptide hybrid molecules bearing LCB1 or other RBD-binding domains, inventors will probe their binding to a homotrimeric spike protein complex (SP3), and use the computational model to guide nanostructure refinement and testing. This trimerized spike protein is available from commercial suppliers, and inventors will rationally design a set of starting designs, approximately positioning the binding peptides to match the position of the ACE2 binding sites on the SP3 (FIG. 5A). Inventors will then use the optimization platform to in silico “evolve” the strongest binder, where the scoring function will optimize both the entropy of binding (by minimizing the entropy loss when the DNA-peptide hybrid molecule is bound to SP3), as well as maximize binding enthalpy; e.g. if the structure is too rigid, the peptides will not be able to correctly dock into the binding site. Given the efficiency of the coarse-grained model, inventors will run thousands of rounds of DNA-peptide hybrid molecule in silico evolution to obtain the most promising candidate nanostructures (˜10-20 total) for experimental testing. For each designed structure to be probed experimentally, inventors will use the model to study its folding to make sure it is able to form correctly, and does not include alternative metastable misfolded states. Preliminary data: LCB1 has been conjugated to DNA handles, and incorporated it into three- and four-helix DNA bundles (FIGS. 5A,B). The monomeric LCB1-DNA conjugate bound equally well as the protein alone (FIG. 5C). Inventors tested these bundles for binding to RBD via an inhibition ELISA assay, whereby RBD binding to immobilized LCB1 competed with soluble DNA-peptide hybrid molecules. Indeed, the trivalent DNA-peptide hybrid molecule bound RBD better than the monomeric LCB1 (FIG. 5D). Although these nanostructures are homo-trivalent, the spike RBD target is still monomeric; experiments are currently underway with the trimeric spike protein (SP3) to directly probe the size-matched binding and affinity enhancement of the DNA-peptide hybrid molecules.

A key feature of multivalent binding is not just enhanced affinity, but a greatly decreased koff for binding, e.g. as seen by Strauch et al. for homotrivalent HA binding proteins. Inventors will probe the binding kinetics of DNA-peptide hybrid molecules by SPR, and compare to nanostructures bearing only one or two peptides/proteins, and mutated (non-binding) molecules. While our model will not be able to directly predict the binding affinity, it will still be possible to rank the structures based on the scoring function. Inventors will compare the experimentally-measured binding affinity with the ranking produced by the model, and seek to adapt the scoring function to match the experiments. Thus, rather than creating a funnel-like approach commonly used in computational design pipelines—where a set of binders is generated, from which only subset is then successfully verified in experiment—our work will create a feedback design loop, where the efficient but coarse model is improved through experimental measurements. At the same time, the model will allow us to effectively search design space and provide iteratively improved designs for experimental probing. As well as determining the affinity between DNA-peptide hybrid molecules and SP3, inventors will also probe the structure's ability to block the spike trimer association with the ACE2 receptor, as a proxy for inhibiting viral infection. In addition to traditional binding/blocking studies via SPR, inventors will also probe the DNA-peptide hybrid molecule binding via negative stain transmission electron microscopy (TEM) and atomic force microscopy (AFM). Both DNA nanostructures and bound proteins can be readily visualized using these methods, so they can be used to demonstrate not just binding, but also affinity (e.g. by counting structures with and without proteins). Results will be compared to free proteins/peptides, and homo-trimerized binding groups using flexible chemical or genetically expressed linkers.

### Determine Distances Between RBD Site and a Separate Binding Site.

Most targets of interests are not homo-oligomeric, so the approach outlined above will not be applicable. Thus, inventors will develop a DNA-peptide hybrid molecule that can position two different targeting groups—where one has a known and the other an unknown binding site on the target—with 3D precision in order to enhance the affinity. Once again, inventors will use the SARS-COV-2 spike protein as the target, because recently several nanobodies, a bispecific IgG mimetic, and an aptamer were reported that did not target the ACE2 binding domain on RBD. One nanobody in particular was shown to bind the N-terminal domain (NTD) of the spike protein, and did not compete with a separate nanobody that bound to the RBD. Inventors will use this NTD site as a test system to (1) develop new computational experimental method that will be able to de novo identify location of binding sites, and (2) create a DNA-peptide hybrid molecule that can position the two groups with spatial precision to match this experimentally-determined distance. Inventors will initially develop a computational-experimental pipeline to determine the location of the second binding site as if the NTD binding site was not known, allowing us to compare our unbiased results to the known location after the fact. The pipeline will generate a set of “nano-rulers,” consisting of the two binding groups linked by simple dsDNA linkers of known length. Inventors will annotate the possible binding sites using available peptides global docking tools that provide a list of approximately 4-10 candidate binding sites, featuring multiple false positives. Inventors will then use the computational platform to design a set of DNA scaffolds with the peptides attached at different distances. Thus, when one peptide (e.g. LCB1) is bound to the RBD, the second peptide on the scaffold covers different distances on the surface of the protein. The set of scaffolds will be designed to cover the respective possible binding distances between the known binding site and the candidate binding site. By comparing the experimental affinity measurements between the designed scaffolds, inventors will be able to select the scaffold that binds to both sites at the same time, and thus “identify” (i.e. confirm) the position on the second binding site (FIGS. 5E,F).

### Design and Test Hetero-Bivalent DNA-Peptide Hybrid Molecules to Match the Distances Determined.

Once the approximate distances and location have been determined for the two binding groups, inventors will design heterobivalent DNA-peptide hybrid molecules that recapitulate this distance and probe for both binding and blocking of the structure to the spike RBD monomer. Although the nanostructures discussed previously can be used, inventors will also explore simpler structure like rigid double-crossover (DX) tiles, where the binding groups can be positioned at multiple locations. The tile will provide added steric bulk for blocking the interaction with ACE2. Inventors will follow the same computational-experimental pipeline as presented previously, and compare the DNA-peptide hybrid molecules to binding groups dimerize using flexible linkers (either alkyl, PEG, or amino acid (via recombinant expression)).

### Demonstrate Stimulus-Responsive “Off” Switch for DNA-Peptide Hybrid Molecule Binding.

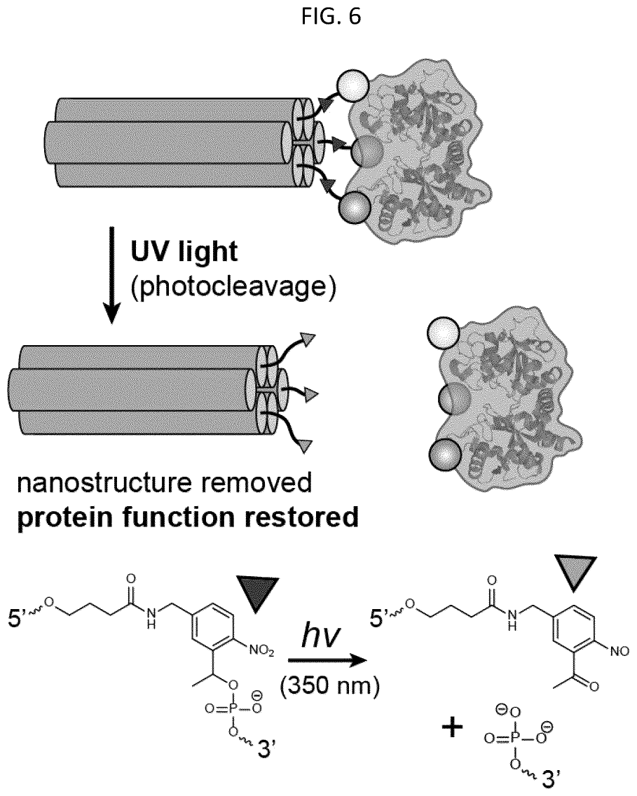

One key advantage of our approach for blocking protein function is that it can, in principle, be reversed by disassembling the nanostructure in a stimulus-responsive fashion. In particular, o-nitrobenzyl ester moieties can be installed into the DNA backbone, resulting in clean scission upon exposure to 350 nm UV light, an approached used by PI Stephanopoulos to install photocleavable functionality into a DNA nanomechanical device. Inventors will incorporate such a photocleavable moiety into the DNA handles attached to the binding groups from presented earlier, so upon UV illumination the entire scaffolding DNA nanostructure is released (FIG. 6). This approach leaves behind the bound peptides/proteins, so it may be necessary to use the lower-affinity binding groups, as opposed to the already high-affinity proteins like LCB1. Earlier, where the binding groups may not target the key protein interface, such a photocleavage will still expose that interface by simply removing the steric blockage imparted by the nanostructure. Inventors will use SPR to probe this UV triggered “activation,” immobilizing the ACE2 receptor on the surface and adding a solution of SP3 pre-blocked using the trivalent DNA-peptide hybrid molecule. Upon UV illumination, the nanostructure should be removed (with a timescale of several seconds for photocleavage, according to our previous work) and the kinetics of ACE2-SP3 binding will be monitored by SPR.

### Expected Outcomes, Potential Pitfalls, and Alternative Approaches:

By the end of this work, inventors will have developed DNA nanostructures bearing: (1) three copies of an RBD-2 binding peptide/protein, or (2) two different binding groups for the spike protein. Inventors will have optimized the computational pipeline to in silico evolve these nanostructures by comparing their bound and unbound state, which is efficient enough to run freely diffusing simulations of the binding trajectory. Potential pitfalls and alternate solutions include the following. (1) Attachment of a DNA handle may compromise peptide binding. While inventors expect that folded proteins like LCB1 will not be greatly affected by DNA handle attachment, it is possible that shorter peptides may be more sensitive helix stabilizing residues or backbone (i, i+7) crosslinks, or use neutral peptide nucleic acid (PNA) handles instead of anionic DNA. (2) The simulations might incorrectly predict the affinity of the designed nanostructures. In that case, inventors will use the experimentally measured affinity to further update the scoring function that will be used in the simulation to assess the enthalpic contribution of binding to the protein surface. (3) The proteins used are too large to effectively position them in 3D space. It is possible that, especially for targeting two different spots on the spike monomer, using LCB1 and a nanobody (or two nanobodies) will be too sterically bulky. In this case, inventors will use cyclic peptides recapitulating the CDR3 loop from the nanobodies, as described in greater length previously. (4) The SARS-COV-2 spike protein is a poor target. If no successful hetero-bivalent DNA-peptide hybrid molecules against the spike protein are found—e.g. because distances determined previously are too small for a DNA nanostructure to effectively bind-inventors will instead turn to a different target: influenza hemagglutinin (HA). Indeed, trivalent protein scaffolds with grafted CDR loops have demonstrated high-affinity binding to this target3, so inventors will use the same loops as starting points for our design. Furthermore, a number of short peptides discovered from on-chip peptide arrays have been reported for HA. Inventors will carry out our “molecular ruler” method for these peptides to find combinations that span distances suitable to DNA nanostructures. Most of these peptides have only modest affinities (Kd˜low micromolar), so attachment to a scaffold could increase the affinity to/past the nanomolar regime, as demonstrated using chemical linkers.

Develop a Photo-Switchable Blocking DNA-Peptide Hybrid Molecule for Fibrinogen. Rationale:

If multiple binding agents are not readily available for a target, one or more must be discovered using selection methods like phage display. However, this approach poses the challenge that the binding sites for these new targeting groups are not known, and thus must be determined prior to incorporation into a scaffolding nanostructure (which will itself be tuned to best recapitulate these distances). Inventors will work with Co-I Stabenfeldt to discover new binding peptides for fibrinogen, in order to block its assembly into fibrin clots. These peptides will not all bind in the same location, so the methods developed in previously will be employed to map their likely distances on the target, in order to design a heterobi- or tri-valent DNA-peptide hybrid molecule that can inactivate the protein-protein interactions. Inventors will also use the photocleavable approach described previously to “turn on” fibrin self-assembly by unblocking the structure. Although inventors will focus on fibrinogen as a proof of principle, inventors will have developed a pipeline for future targeting of any protein through a three-step process: (1) Identify a subset of binding nanobodies/peptides against the target; (2) Determine the pairwise distances for proteins/peptides that bind to nonoverlapping sites; and (3) Design a DNA-peptide hybrid molecule to effectively envelop the target, using the computational experimental approach outlined earlier.

### Preliminary Data:

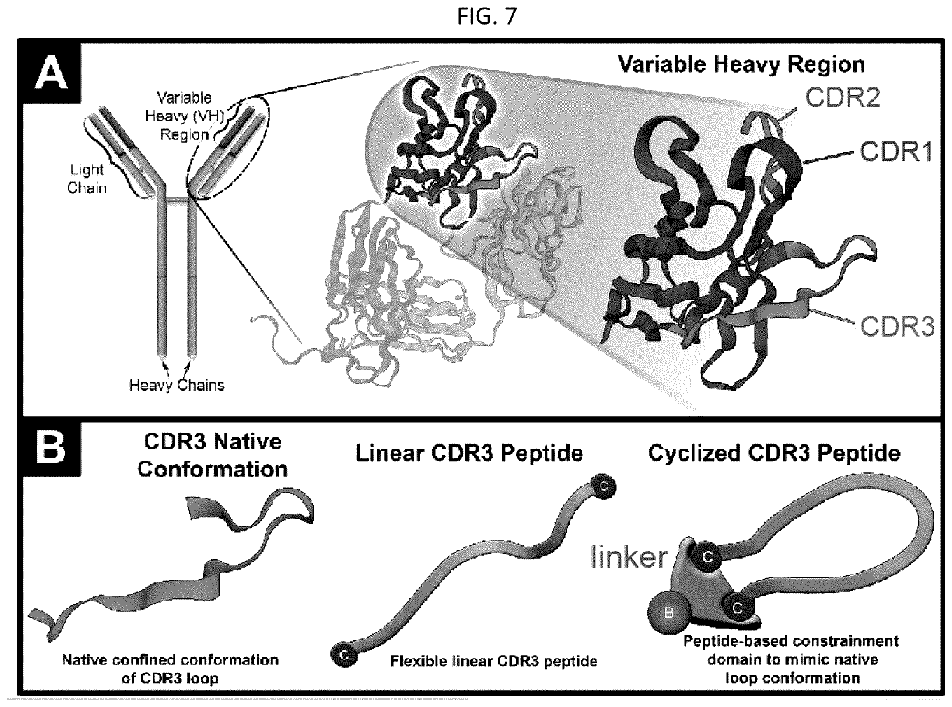

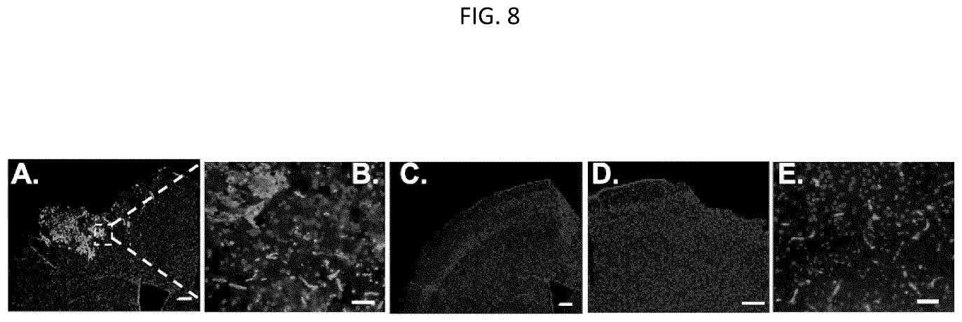

Phage display can be used to find novel targeting nanobodies against complex targets such as fibrin, in vitro cell culture models of reactive astrocytes, ex vivo tissue sections from small and large animal models of brain injury, and in vivo brain injury mouse models. However, the target nanobodies are often difficult to express recombinantly, leading to poor yields or aggregation. Thus, it was recently reported that cyclized peptides from the CDR3 loop of targeting nanobodies can be highly effective as targeting agents, while retaining a small size and ease of synthesis. This approach was termed the CDR3 Loop Assembly via Structured Peptide (“CLASP”) system (FIG. 7). Inventors demonstrated such power of phage display by identifying CDR3 motifs with a domain antibody phage library (dAb) that recognize temporal alterations in the neural injury microenvironment (FIG. 8). Inventors conducted three in vivo phage biopanning screens with the dAb phage library in mice that sustained a focal TBI (controlled cortical impact; CCI) at three different time points post-injury (1, 7, and 21 days post-injury; dpi). Using next generation sequencing and bioinformatics analysis, inventors then compared and identified enriched phage populations for each time point post-injury (FIG. 8). The bioinformatic analysis focused on ranking by CDR3 as this region imparts high diversity and specificity for dAb/antigen recognition compared to CDR1 and CDR2. This analysis pipeline enabled selection of prominent CDR3 targeting domains for either acute injury (1 dpi) and subacute (7 dpi). The discovery was further made possible by applying strict selection criteria to identify top candidate CDR3 sequences for further characterization for each time point. The selection criteria included: (1) unique to a distinct temporal phase post-injury, (2) not present in control phage libraries (amplified without biopanning), or peripheral tissue (heart, liver, spleen), or sham library, and (3) high frequency and enrichment observed round to round. After applying this selection criteria, inventors used the CLASP system to generate CDR3 mimetics for validation testing (FIG. 7B). Ultimately, inventors successfully identified and validated two CLASP cyclic peptides that recognize acute (1 dpi) or subacute (7 dpi) TBI. The immunohistochemical based assessment on post-mortem murine TBI tissue presented in FIG. 8 demonstrate the stark temporal and spatial localization to neural injury by the acute and subacute CLASP motifs. By using synthetic peptides, it will also be possible to explore nanostructure design and tighter integration of the peptides into the DNA scaffold to better mimic loop placement on antibodies. Here, in this application, inventors will leverage extensive experience with fibrin/fibrinogen targeting and polymerization dynamics to focus on fibrin as a proof of principle to develop a pipeline for future targeting of any protein of interest.

### Phage Display Against Key Fibrinogen Polymerization Domains to Discover Nanobody CDR3 Loops.

Inventors will leverage prior knowledge of the fibrin knob-pocket interactions that drive fibrin assembly and polymerization; specifically, inventors will use the short peptide sequence of GPRPXX (SEQ ID NO: 3) that recognizes hydrophobic pocket domains on the beta and gamma chains. Phage display with the aforementioned dAb phage library against fibrinogen in the presence of the GPRPXX (SEQ ID NO: 3) peptide (at millimolar concentrations to compensate for its modest Kd (5-10 μM) will be conducted to identify recognition domains outside of the pocket regions. Human fibrinogen will be immobilized on microbeads via EDC/NHS chemistry. Inventors will carry out biopanning with a naïve human dAb phage library, which will be produced and purified per protocol. Substrates will be incubated with dAb phage (100 μl of 1010-1012 CFU) for 1 hr. Non-specific binding phage will be removed via a series of rinses with PBS+0.1% Tween 20 (PBST). The target bound phage will then be eluted, collected, and amplified. Subsequent rounds will be repeated with an enriched population of eluted phage from the previous round. A minimum of three biopanning rounds will be completed, with a goal of obtaining 10-20 nanobodies that span a range of binding areas on the protein. To identify the CDR3 loop, inventors will carry out next generation sequencing (NGS) and bioinformatic analysis. The use of NGS provides a robust and high-throughput alternative to Sanger sequencing with extensive coverage, enabling an in-depth analysis on the eluted phage libraries. Here, amplified plasmid DNA from the eluted phage libraries will be prepared for Illumina MiSeq 2×250 sequencing. Paired end sequences will be stitched together using Fast Length Adjustment of SHort Reads (FLASH). HCDR3 sequences will be clustered using a hierarchical Levenshtein Distance algorithm with FASTApatmer Perl scripts. Each library will be searched for HCDR3 sequences that are enriched through the biopanning round using a combination of in-house R scripts and Galaxy modules. The top enriched dAb sequences will be selected based on the HCDR3 analysis and the following selection criteria: 1) unique to a distinct target, 2) not present in control phage library (amplified without biopanning), and 3) high frequency and enrichment observed round to round.

### Synthesis of Cyclic Peptides and DNA Conjugates.

Following identification of nanobody-derived CDR3 loops that bind to fibrinogen, inventors will next synthesize cyclic version of these peptides by introducing terminal cysteine residues and bis-bromoacetamide linkers as described previously. The linkers will also incorporate linear alkynes for copper-catalyzed click coupling to azide DNA. Peptide-DNA conjugates will be purified and characterized as described in earlier, and individually tested for binding to fibrinogen by SPR (both cyclic peptides alone and DNA conjugates thereof).

### Determination of Pairwise Distances for Three-Peptide Sets.

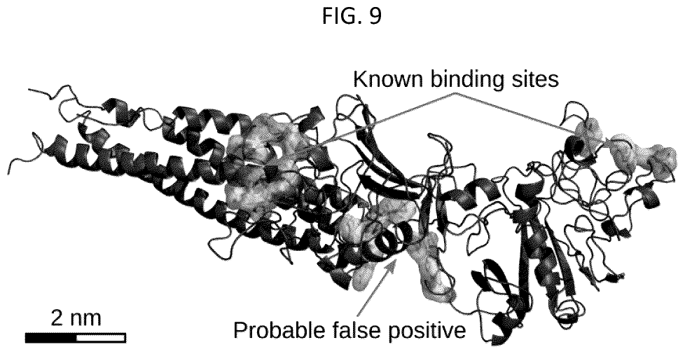

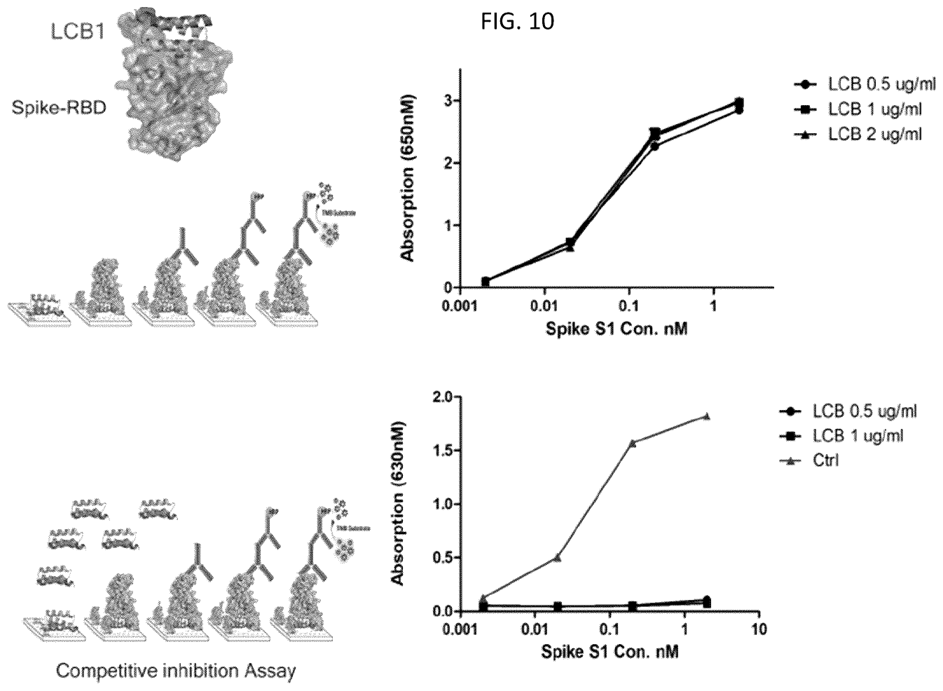

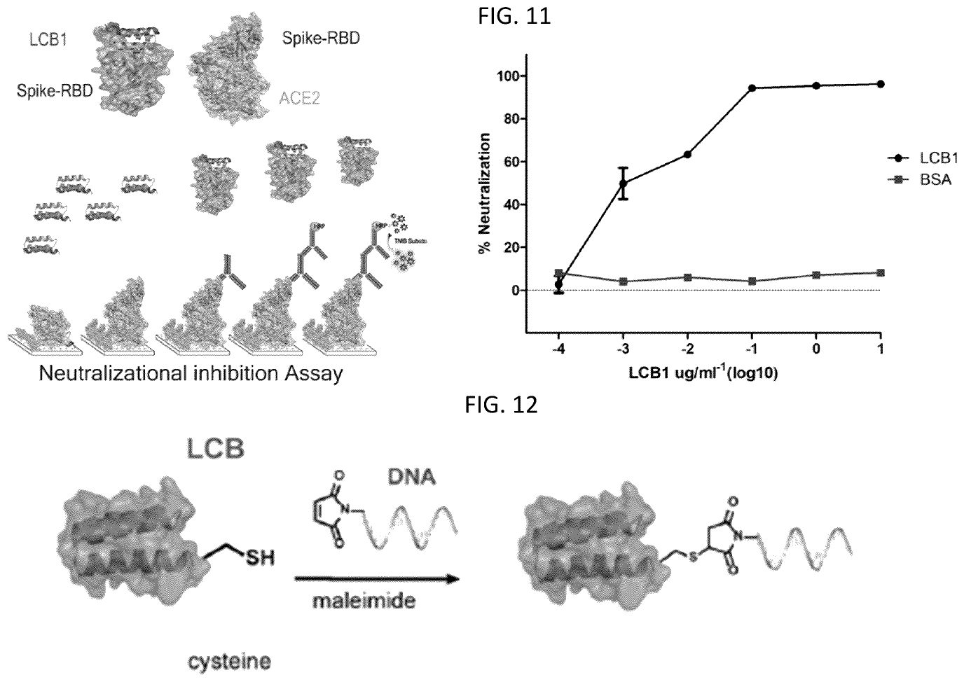

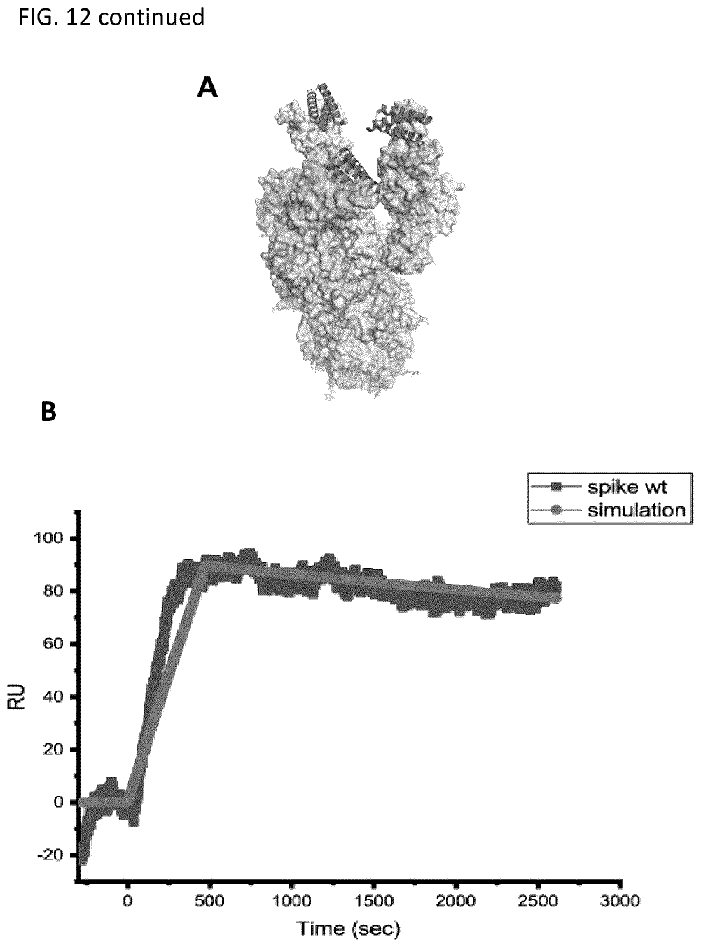

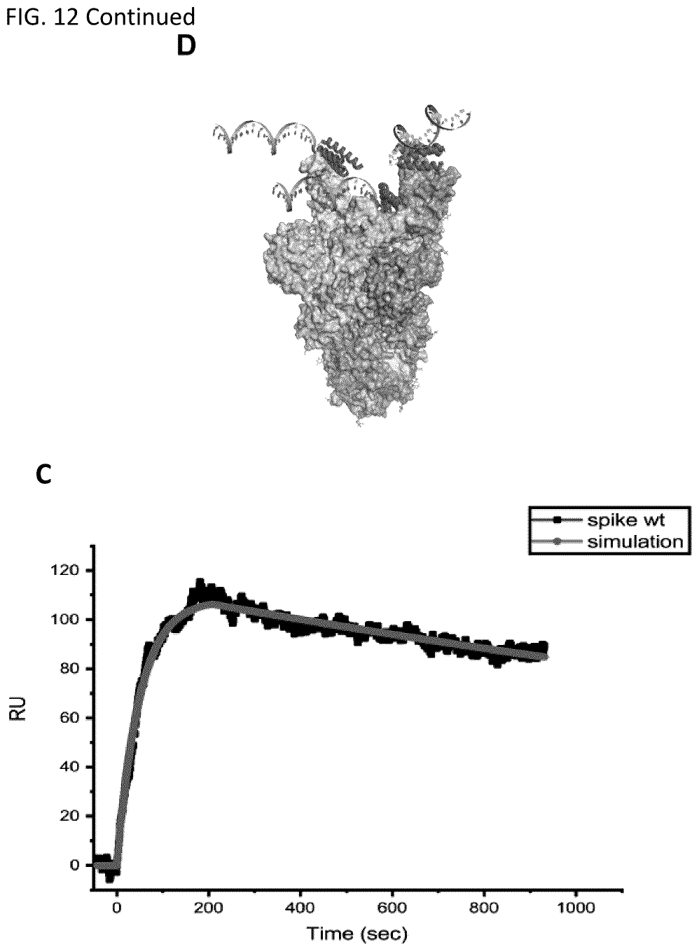

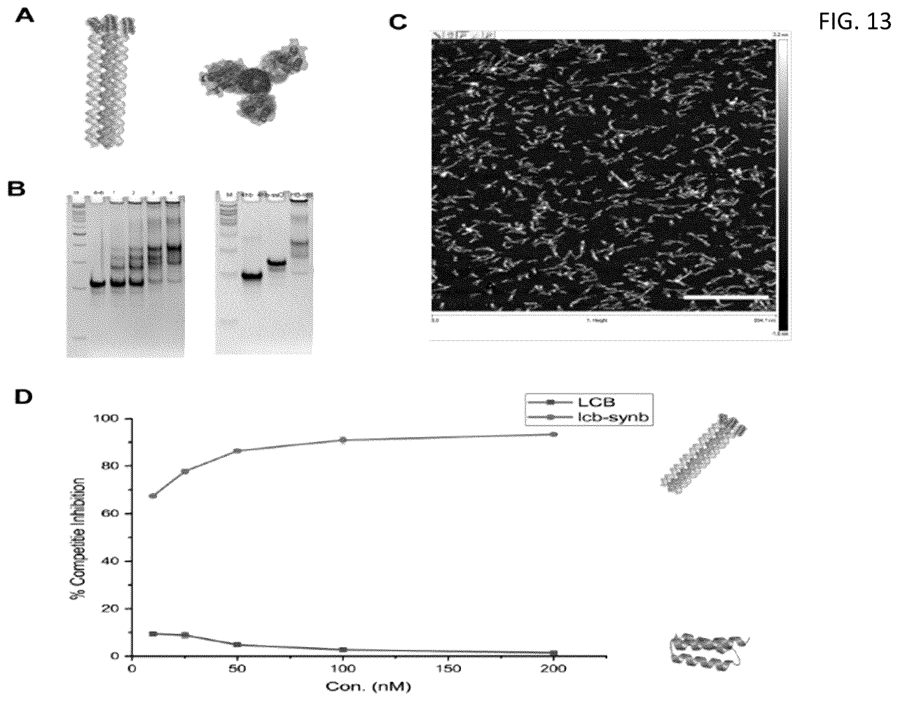

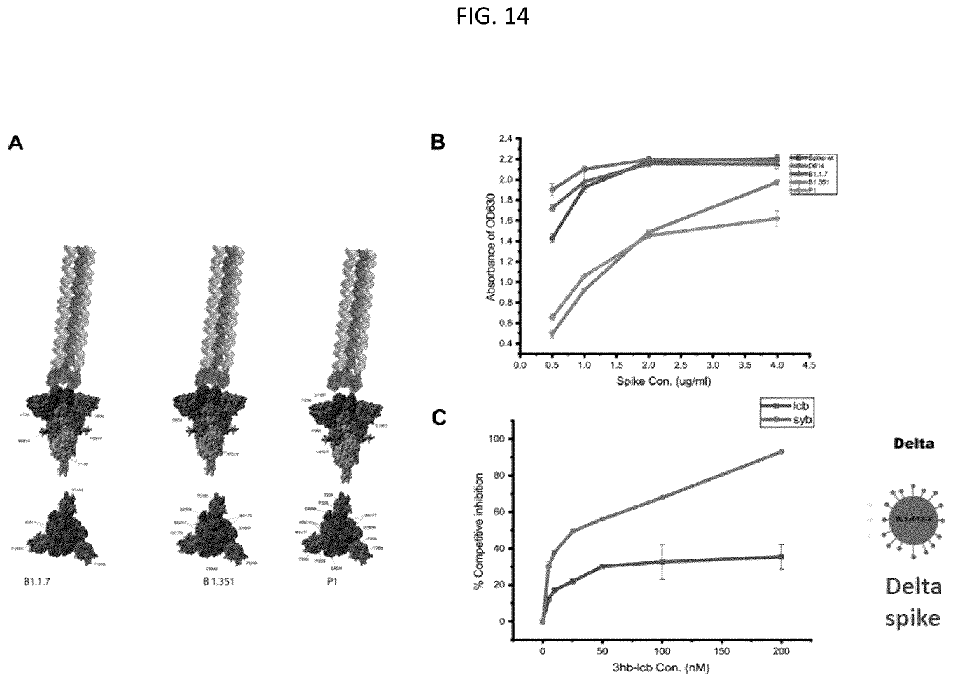

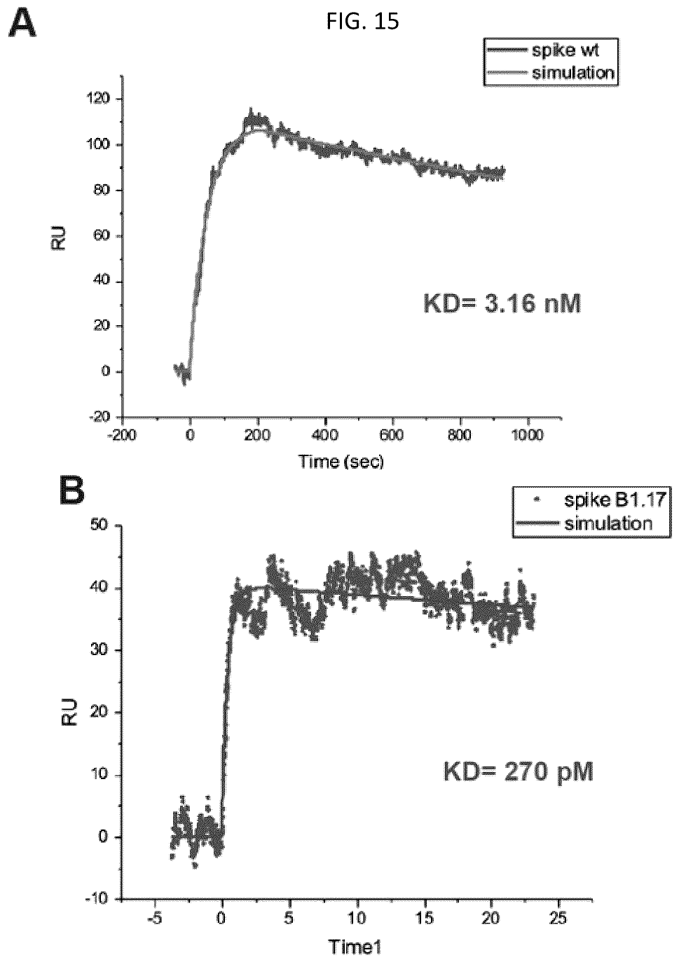

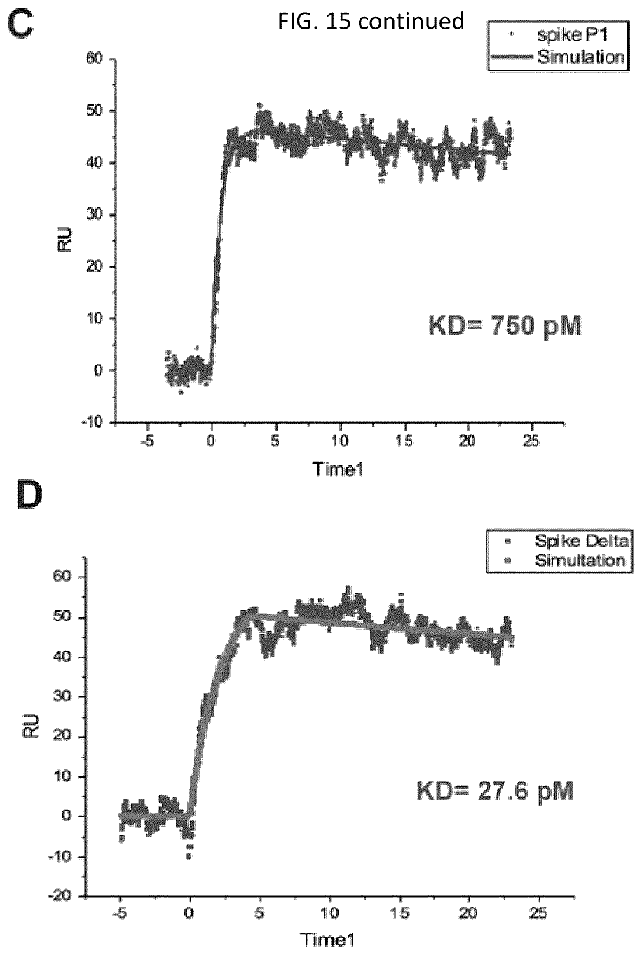

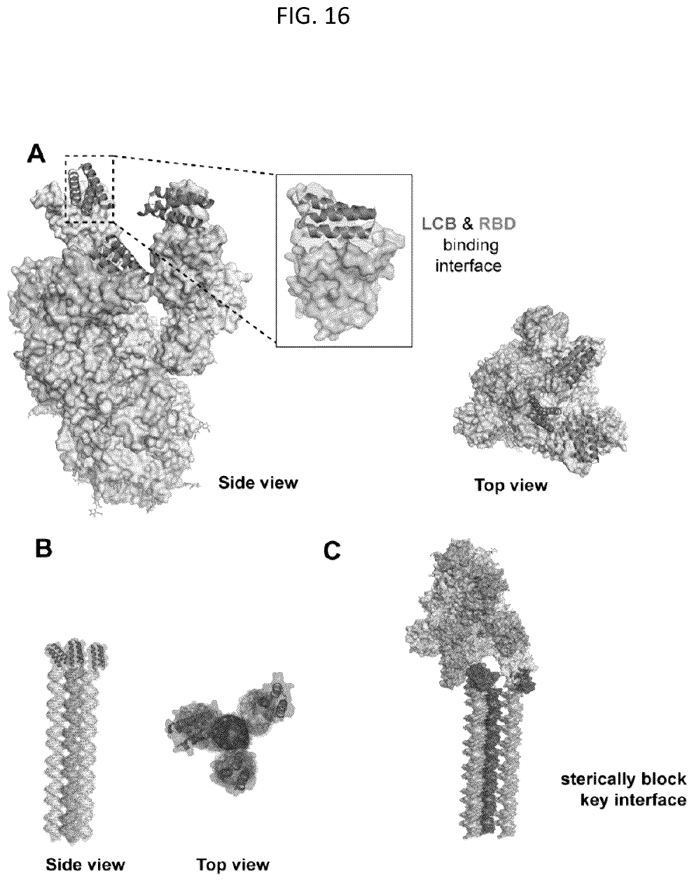

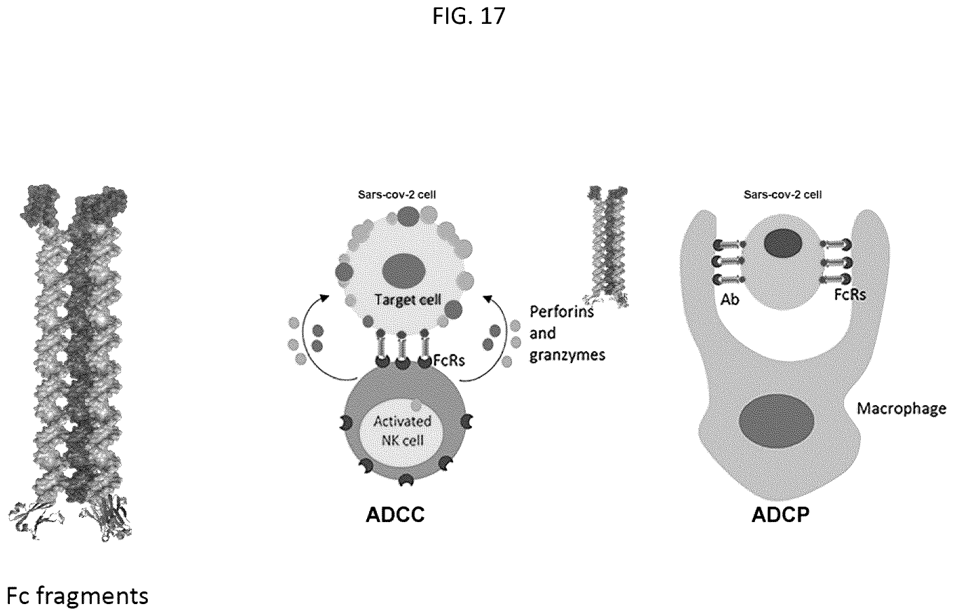

Inventors will next use the set of peptide-DNA conjugates to map out potential binding sites to fibrinogen, as outlined above. One of the peptides used will be the GPRPXX (SEQ ID NO: 3) sequence that binds to the pocket domains, and it will serve as a way to “pin” the possible distances covered by the other peptides. Following the experimental-simulation pipeline developed in a first aspect, inventors will use available global docking tools to annotate likely binding sites for the peptides identified to bind to fibrinogen in the phage display experiments (FIG. 9), and will develop a set of DNA “nanorulers”, where one end functionalized with GRPRXX (SEQ ID NO: 3) peptide binds to the pocket domain, and the other ends are designed to bind to one of the candidate binding sites. Using in silico evolution, inventors will develop the rulers to only bind to the known and candidate sites, and test the set of nano-rulers in experimental measurement in affinity, identifying the ones with the highest affinity that correspond to the nano-ruler binding the GPRPXX (SEQ ID NO: 3) binding site and the candidate site on fibrinogen.

### Design and Testing of Hetero-Trivalent DNA-Peptide Hybrid Molecules.

Inventors will use in silico iterative evolution framework developed in earlier to design candidate DNA nanostructures that position the peptides in 3D space. These structures will constrain GPRPXX (SEQ ID NO: 3) and, ideally, two additional CLASP peptides at the distances determined previously, and binding to fibrinogen will be probed using SPR as described in a first aspect. In addition, inventors will probe the functional blocking of fibrin polymerization by the nanobody, following proteolytic cleavage by thrombin, using a suite of fibrin polymerization assays. Specifically, inventors will assess polymerization dynamics (turbidity and thrombin clotting time), extent of clottable protein, and clot structure (confocal microscopy). Fibrin polymerization assay: Thrombin-initiated fibrin polymerization assays will be used to evaluate anticoagulant activity. For all assays, fibrin clots will be prepared with final concentrations of human fibrinogen at 1 mg/mL (plasminogen-, fibronectin-, von Willebrand Factor-depleted), human α-thrombin at 1 NIH U/mL (ERL), activated human factor XIII at 1 U/mL in a HEPES-buffered solution supplemented with calcium chloride. Prior to initiating polymerization, 50 μL of fibrinogen or fibrinogen+hetero-trivalent DNA-peptide hybrid molecules will be incubated at room temperature for 30 min in a transparent 96-well plate. Polymerization will be initiated by adding 50 μL of thrombin+FXIIIa to each well. Turbidity curves will be generated from absorbance measurements recorded every minute for 60 min at 350 nm. Post-assay analysis of turbidity curves will include the peak absorbance and thrombin clotting time. Percent clottable protein: Upon completing turbidity assays, the resulting fibrin clots will be removed, leaving behind the remaining soluble protein (i.e., the clot liquor). The soluble protein content in the clot liquor will be quantified using a Quant-iT protein assay (Invitrogen). Data will be assessed as percent clottable protein, the amount of initial protein minus soluble protein in the clot liquor all divided by the initial protein. Fibrin fiber structure: Confocal microscopy will be used to evaluate the fibrin fiber structure. Briefly, fibrin clots will be prepared as described above with addition of 5% fluorescently labeled fibrinogen. Upon initiating polymerization with thrombin and FXIIIa, 100 μL will be immediately transferred to a glass slide with 300 μm spacers and capped with a cover slide. Clots will be imaged 60 min after polymerization. Five random 10 μm zstack sections of each clot will be imaged with a Zeiss Laser Scanning Microscope. Image analysis and 3D projections will be performed with ZEN imaging software.

### Reversible Blocking of Fibrin Assembly.

As outlined above, an advantage of our approach is the ability to cleave the nanostructure for the binding peptides/proteins using UV light. Given that some of the peptides discovered herein will bind to sites away from the key binding interface, it is likely that photo-removal of the DNA scaffold will restore binding even if the individual peptides remain bound. In particular, the weak (micromolar) affinity of GPRPXX (SEQ ID NO: 3) for the pocket suggests that upon nanostructure cleavage, this peptide will dissociate from the protein without the avidity effects of the other binding groups. Pre-blocked fibrin will be cleaved using thrombin as above, and then exposed to UV light to remove the DNA-peptide hybrid molecule. The kinetics of polymerization will be compared with unblocked controls, and the fibrin fibers examined.

### Expected Outcomes, Potential Pitfalls, and Alternative Approaches:

Inventors will have developed a novel DNA-peptide hybrid molecule that positions up to 3 cyclic peptides derived from phage display to block fibrin assembly until activated using light. Potential pitfalls and alternate solutions include the following. (1) Phage display against the fully intact fibrinogen protein does not yield relevant CDR3 domains. If the CDR3 domains cannot block polymerization, inventors will use enzymatically or chemically cleaved fragments of fibrinogen to further refine/constrain the target to the pocket domain (i.e. fragment D). (2) Global docking tools are unable to give sufficient number of candidate binding sites and none of the designed nano-rulers can successfully identify the binding sites of the CD3 loops. In such case, inventors will design in silico a set multivalent nanostructure functionalized with CDR3 loops selected against chemically cleaved individual fragments of fibrinogen. Inventors will optimize the nanostructure so that its respective arms with attached CD3 loop are designed to cover the entire protein fragment against which the CDR3 loop was selected. (3) DNA conjugation perturbs cyclic peptide binding affinity. If the DNA handles reduce or abolish the CLASP peptide binding, inventors will explore constructs with varied linker lengths, or use PNA handles instead of DNA to avoid charge repulsion. It may also be necessary to append both ends of the peptide directly to the DNA backbone (using the structure to effectively cyclize it) in order to reduce flexibility in the system.

The two broad aspects of the disclosed technology outlined above rely on knowing individual binding proteins or peptides. A more powerful method, however, would be to directly select the bi- or tri-valent nanostructure, by creating a combinatorial library of all possible peptide/protein combinations on the scaffold. Inventors envision creating libraries of formed nanostructures with random combinations of peptides/proteins and selecting for the final assembly. For instance, peptide-RNA conjugates generated from mRNA display can be integrated into the scaffolds through a common poly(A) linker. However, the nanostructures used to scaffold these peptides/proteins will be tunable from the outset to match the rough size of the target, and a subsequent optimization of the scaffold could be performed to further enhance binding. Following selection of the best heterotrivalent nanostructure, the peptide identity can be deduced via sequencing of the appended mRNA handles. Finally, our approach can be used to block previously un-targetable proteins; by using any surface on the protein as a “handle” to help associate a nanostructure and block a key interface, inventors expand the space of targetable protein patches. The use of multiple binding sites to enhance affinity can also reduce mutational escape if any patch changes, and allow the combination of peptides, aptamers, and even small molecules on the scaffold.

### Example 2

### Tri-Pdbody for SARS-Cov-2 Therapeutics

Severe acute respiratory syndrome coronavirus 2 (SARS-Cov-2), a novel coronavirus first identified in December 2019, is the causative agent of coronavirus disease 2019 (Covid-19). The SARS-Cov-2 infection generally begins in the nasal cavity, with the virus replicating there for three days before spreading to the lung, brain, and accompanying organs by widespread virus replication in the gut and genital tract. SARS-Cov-2-neutralizing monoclonal antibodies (NAbs) are an attractive treatment and can be used both in COVID-19 prevention and therapy. A recent study showed that an antibody cocktail approach, which is made up of two non-competing, neutralizing human IgG1 antibodies that target the receptor-binding domain (RBD) of the SARS-COV-2 spike protein, provided a profound survival benefit for patients infected with SARS-COV-2. The antibody cocktails prevented viral entry into human cells through the angiotensin-converting enzyme2 (ACE2) receptor.

However, SARS-Cov-2 is a member of the coronavirus family, which carries the largest genome among single-stranded RNA viruses that mutate frequently. Some recent reports showed the multiple variants emerging around the world as the duration of the SARS-Cov-2 pandemic extends, such as Covid-19 alpha (B1.1.7) from the UK, Covid-19 beta (B1.351) from South Africa, Covid-19 gamma (P1) from Brazilian and Covid-19 delta (B1.617.2) from India, and so on. These lineages are characterized by numerous mutations in the spike protein, raising concerns that they are not affected by neutralization monoclonal and vaccine-induced antibodies. It is, therefore, urgent to develop de novo molecules targeting viral spike variants RBD for SARS-Cov-2 therapeutics.

### Trivalent Protein-DNA Synbody (Tri-Pdbody) Neutralization Against any SARS-COV-2 Spike Variants

### Tri-Pdbody Design

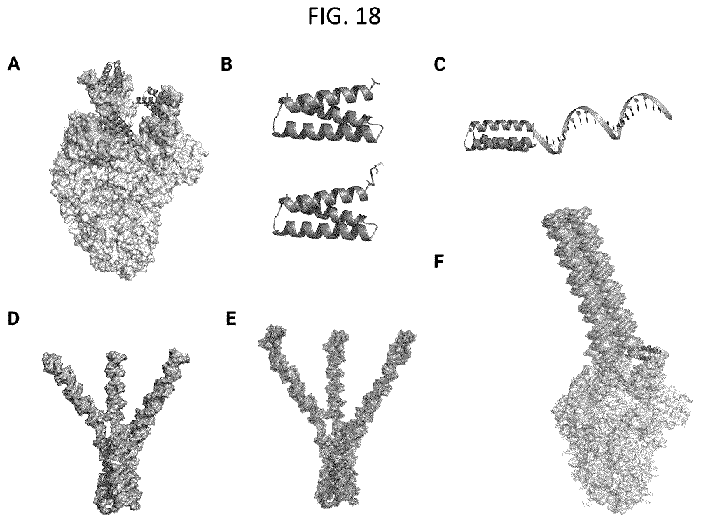

The ability to target the receptor binding domain (RBD) of SARS-COV-2 spike with high specificity and efficiency is critical to the neutralization of SARS-COV-2 virus to prevent infection. DNA has emerged as an exceptional molecular building block for functional molecule assembly due to its predictable confirmation and programmable intra-molecular Watson-Crick base-pairing interactions. Based on the spike trimer geometry structure and de novo mini-binder, inventors have designed a trivalent DNA-protein hybridized nanostructure with high-affinity binding with spike proteins. Our preliminary data shows that the mini-binder has high-affinity binding with spike protein and variants (KD˜pM). Thus, inventors propose to build a tri-valent DNA-mini-binder hybridized nanostructure (tri-pdbody) to specifically target the three RBD of spike trimer protein and variants. As shown in FIG. 18, based on spike trimer protein geometry, inventors choose mini-binder conjugated with DNA as a target binding site, which assembles three mini-binders on a four-helix bundle DNA structure as a trivalent DNA-mini-binder nanostructure.

Minibinder Conjugated with DNA

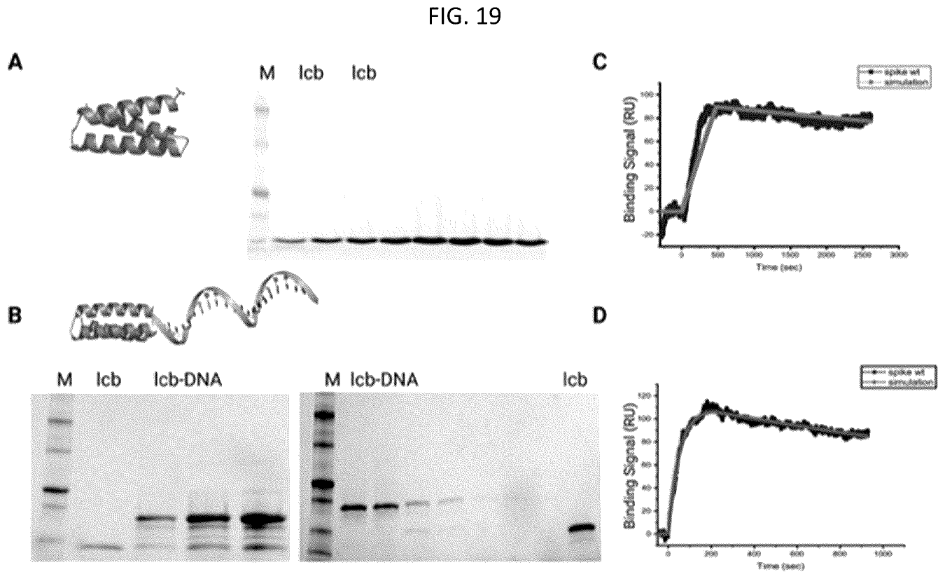

To conjugate with DNA for mini-binder, inventors replaced the last codon as a cystine which the thiol group can be easy for modification with DNA. Inventors mutated the last residue to cysteine (S78C) in order to carry out thiol-selective chemistry with the sulfo-succinimidyl 4-(N-maleimidomethyl) cyclohexane-1-carboxylate (Sulfo-SMCC) and a 14-nt 5′ amino modified oligonucleotide (FIG. 19). To assess the binding affinity of wild type mini-binder and mini-binder with DNA against spike protein, the wild type spikes were immobilized on gold films mini-binder/mini-binder with DNA as a solution to evaluate its binding association and dissociation using surface plasmon resonance (SPR), shown in FIGS. 19 C, D.

### Minibinder Binding Specificity and Avidity of Different Spike Variants

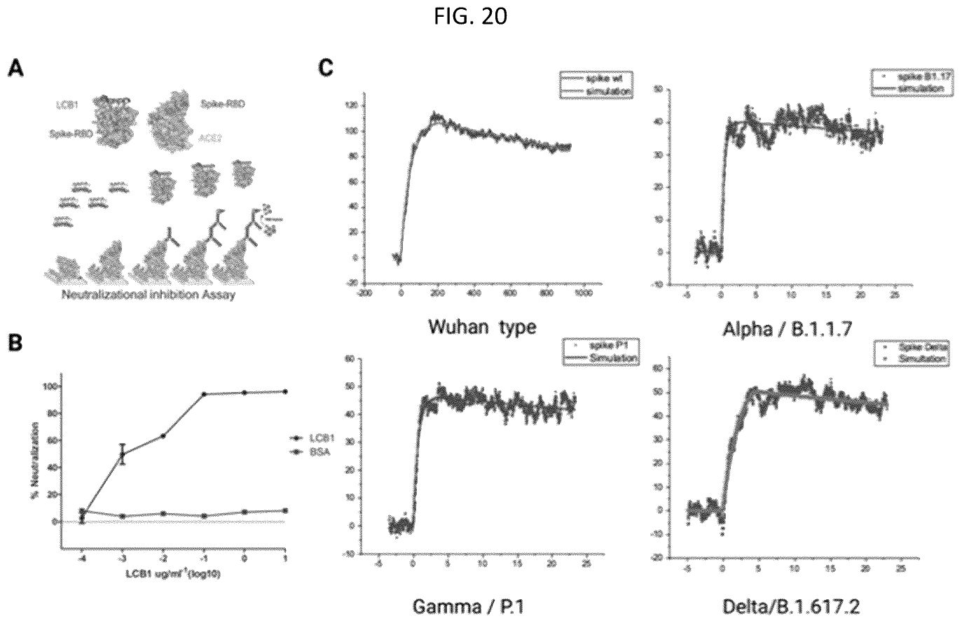

To assess the specificity of mini-binder, the recombinant ACE2 protein were coated on the 96-well plate, the mini-binders and ACE2 protein were competitive binding with RBD of spike protein, schematic shown in FIG. 20A. Inventors found the mini-binder completely neutralized the RBD binding with ACE2 protein, compared with Bovine serum albumin (BSA) protein which was non-competitive binding signal, indicated that the mini-binder can strongly bind with RBD of spike and competitive inhibition of ACE2 binding with the RBD of spike protein. To assess the mini-binder binding with other variants, the wild type spike and other variants spike were immobilized on gold films individually, mini-binder as a solution phase to evaluate its binding affinity SPR, shown in FIG. 20C.

### Different Arm Tri-Pdbodys Assembly

To assemble with different arm tri-pdbodys, inventors designed one, two, three arm 3 helix bundle (3HB) DNA nanostructure, shown in FIG. 21A. the one, two, and three arm 3HB were characterized by PAGE and imaging by AFM (FIGS. 21BC). To assess the effect of tri-pdbody assembly, the one, two, and three arm 3HBs were mixed with mini-binder with DNA respectively. Specifically, the one arm 3HB added the equivalent mini-binder with DNA, the two arm 3HB added the 2 folds mini-binder with DNA, the three arm 3HB for three folds mini-binder with DNA. The assembly mono-, bi- and tri-pdbody was evaluated by gel assay and imaging by AFM. Our data indicates that our design can be easy to assemble to form the different arms 3HB, and assembly with mini-binder as the different valent synbody.

### Measurement of Binding Kinetics of Different Arm Tri-Pdbody

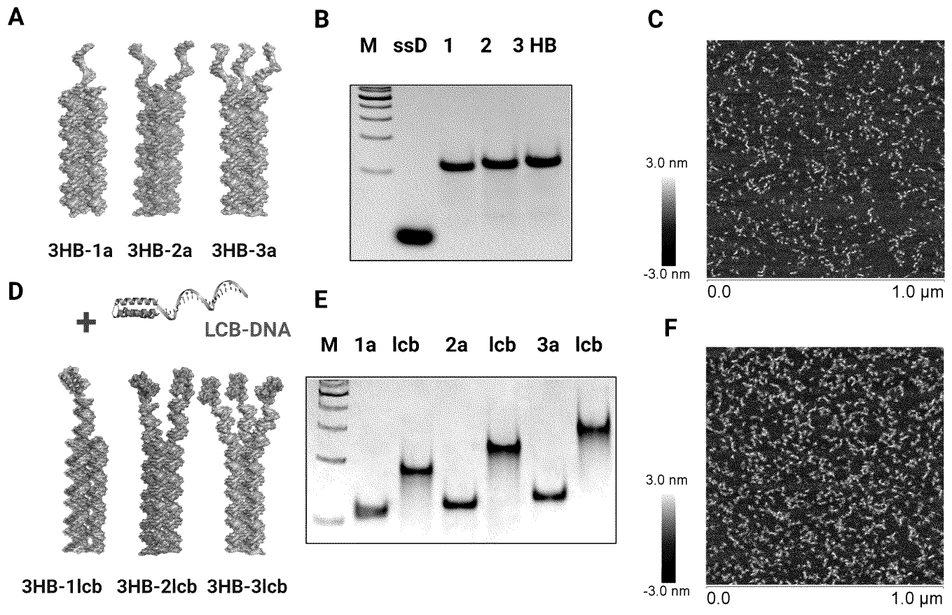

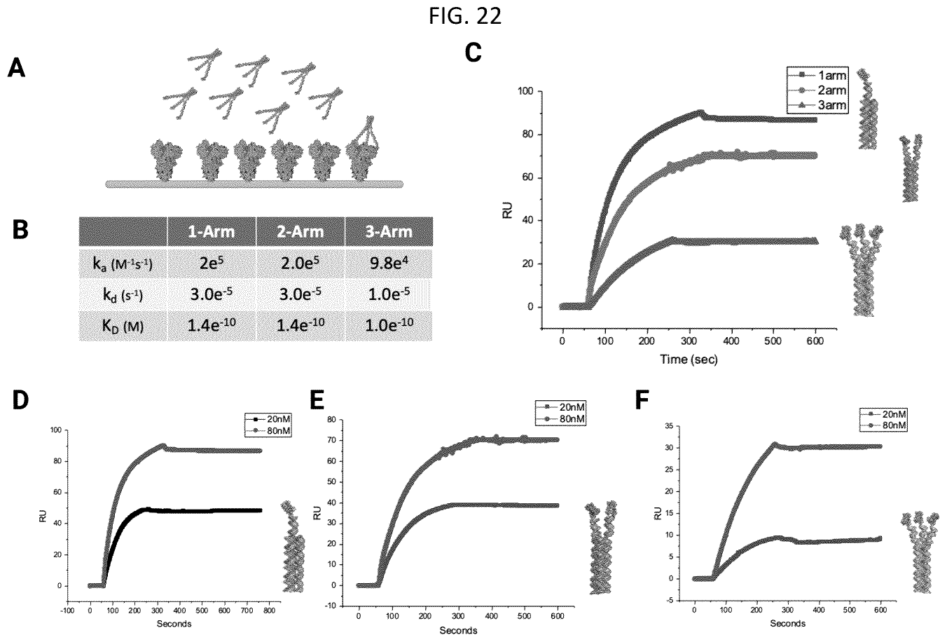

To assess the binding kinetics of the mono, bi, and tri-pdbody, inventors immobilized the recombinant Delta spike protein on the functionalized CM dextran chip, and measured the binding kinetics of the mono, bi-, and tri-pdbody respectively (FIG. 22). Initially, inventors flowed PBS buffer (10 mM) at a rate of 60 ul/min over the dextran chip and recorded the response signal (RU) to establish a baseline, and then introduced each of the mono, bi-, and tri-pdbody to allow binding (association) to the corresponding target the Delta spike protein on the dextran chip. After measuring the association process, inventors studied the dissociation of the tri-pdbody from spike by flowing PBS buffer over the dextran chip and observed that the amplitude didn't return to prebinding levels. Our data confirmed that the specific binding of mon, bi-, and tri-pdbody strongly corresponded delta spike protein.

### Measurement of Binding Kinetics of Tri-Pdbody and Antibody

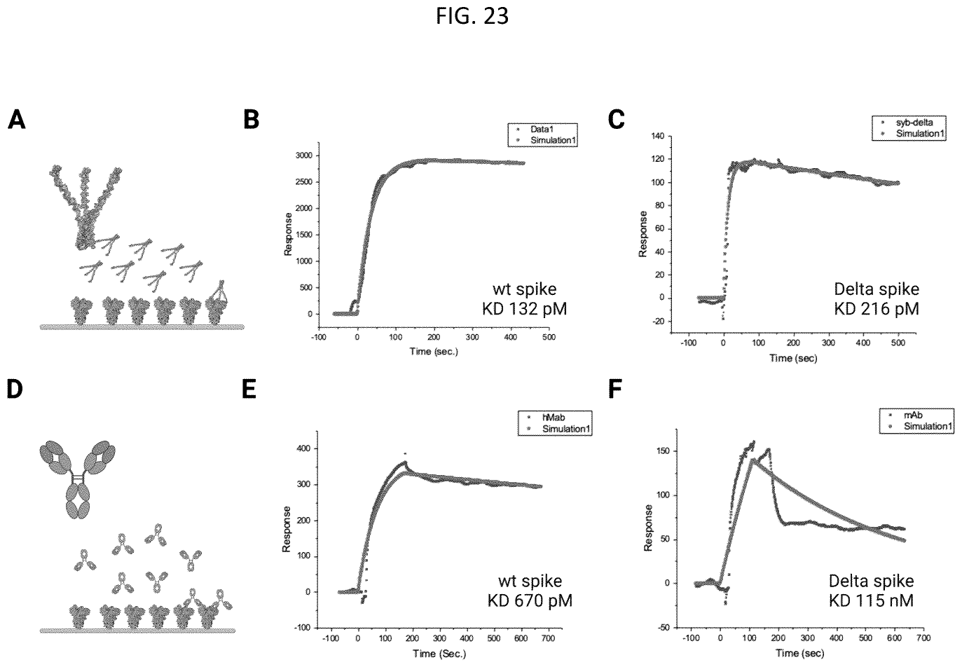

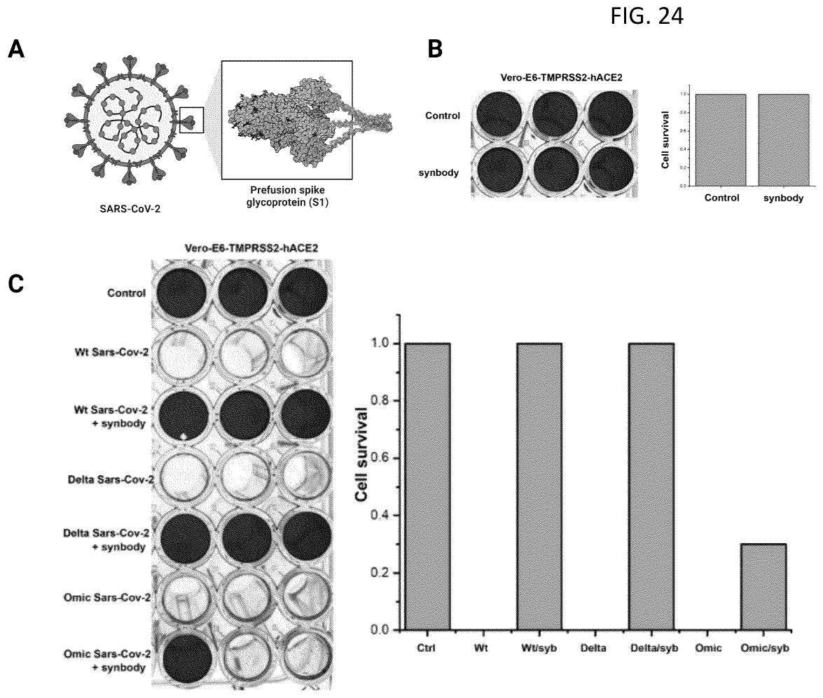

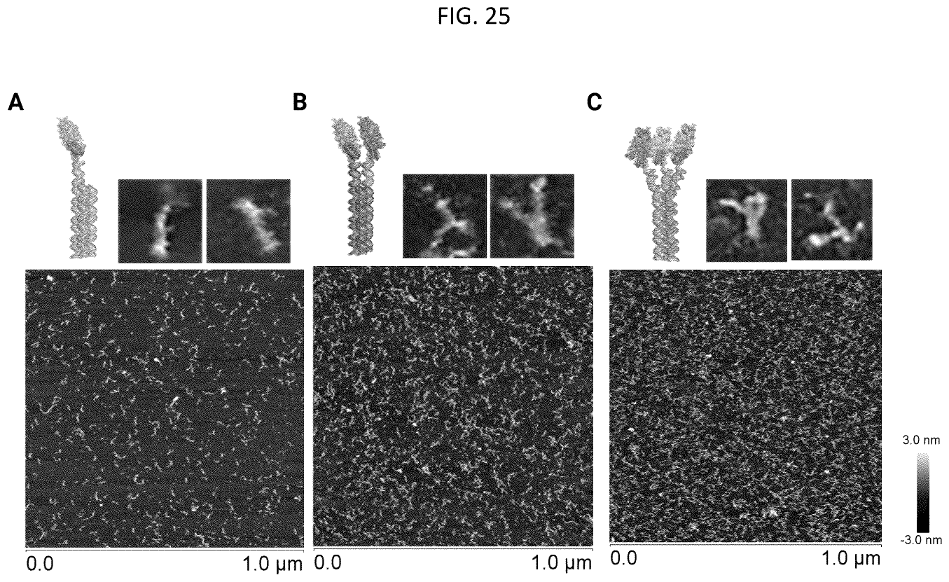

To confirm the binding kinetics of the tri-pdbody and commercial antibody, inventors immobilized the e recombinant Delta spike protein on the functionalized CM dextran chip and measured the binding kinetics of the tri-pdbody and commercial antibody respectively (FIG. 23). Initially, inventors flowed PBS buffer (10 mM) at a rate of 60 ul/min over the dextran chip and recorded the response signal to establish a baseline, and then introduced tri-pdbody and antibody to allow binding (association) to the corresponding target the wild type spike and delta spike protein on the dextran chip respectively. After measuring the association process, inventors studied the dissociation of the tri-pdbody/antibody from spike by flowing PBS buffer over the dextran chip and observed that the amplitude didn't return to prebinding levels. Our data confirmed that the specific binding of tri-pdbody strongly corresponded wild type and delta spike protein, but the commercial antibody only corresponded the wild type spike protein.

### Anti-SARS Cov-2 Virus Infection Assay

Since SARS-Cov-2 is known to mutate in order to avoid the increased risk of mutation-induced neutralization escape, it is vital to apply a tri-pdbody neutralization against virus infection. The neutralization efficiency was investigated by determining the cell survival with or without treatment of individual tri-pdbody. As expected, the tri-pdbody showed the highest neutralizing efficiency compared to the nucleic acid structure without tri-pdbody treatment. The tri-pdbody neutralized SARS-COV-2 virus, resulting in the survival all the cells. All cells were dead after SARS-Cov-2 infection without the neutralizing tri-pdbody.

The Assembly of Mono, Bi, and Tri-Pdbody Binding with RBD

To determine whether the mono-, bi-, and tri-pdbody can bind with the receptor binding domain of spike protein, inventors assembled the mono-, bi-, and tri-pdbody. These different arms were mixed with RBD protein with equivalent, 2 fold and 3 folds ratio. Data showed the one arm, two arm, and three arm state of the synbody with RBD.

Synbodies are Capable of Binding with Delta Spike and Omicron Spike

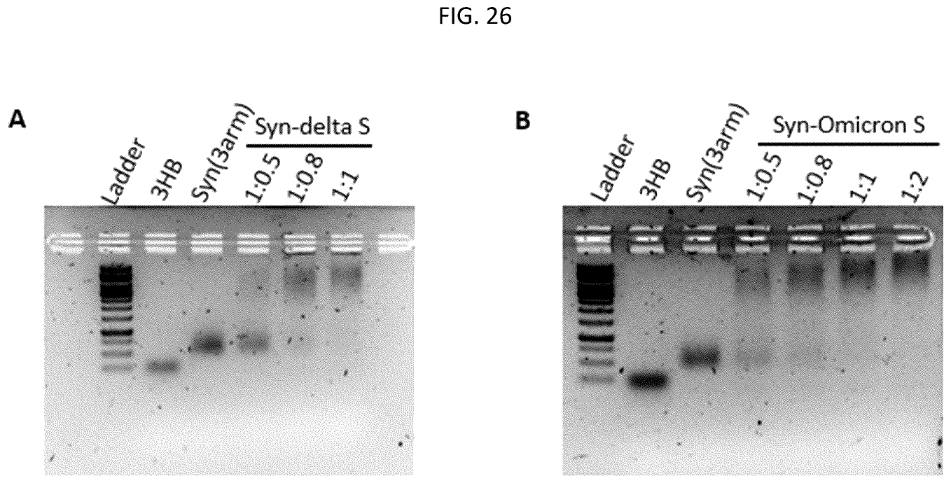

In previous experiments the inventors showed that synbody binds to wild-type spike proteins at pico-molar levels. Accordingly, the inventors tested the spike protein binding ability of synbody with other SARS-COV-2 mutant strains. As previously described, first 3-HB DNA nanostructures were assembled with three DNA-LCBs to form the synbody. Next, different molar ratios of synbody were incubated with delta spike and Omicron spike, respectively, for 10 mins at 37° C. And then, the binding was characterized by agarose gel electrophoresis to detect DNA migration. As shown in FIG. 26, after incubation with spike protein, significant DNA band migration was observed, indicating that synbody binds well to both delta spike and Omicron spike.

### Characterization of SARS-COV-2 Pseudovirus

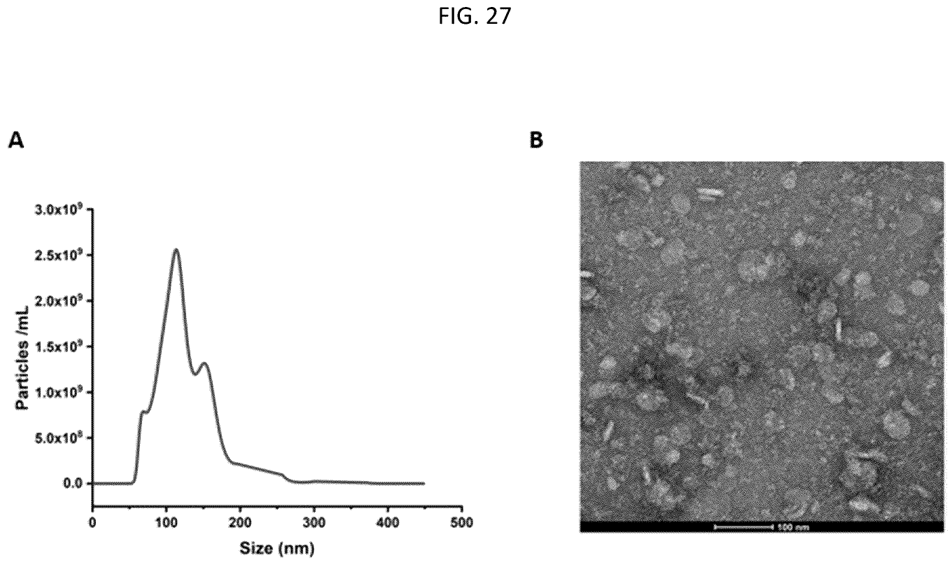

To verify the neutralization ability of synbody against SARS-COV-2, inventors constructed a SARS-COV-2 pseudovirus system. Briefly, HIV lentiviral vector, spike expression vector and GFP expressing lentiviral vector were used to co-transfect into Plenti-X cells, and the virus was collected from the supernatant after 48 hours and concentrated by PEG precipitation. Next, inventors analyzed the size of the pseudovirus particles using NanoSight NTA, which showed an average size of 109 nm, consistent with the size of the HIV backbone pseudovirus particles. In addition, pseudovirus particles were also observed under Negative stain EM. (FIG. 27)

### Neutralization of Wild-Type Pseudovirus Infection by Synbody

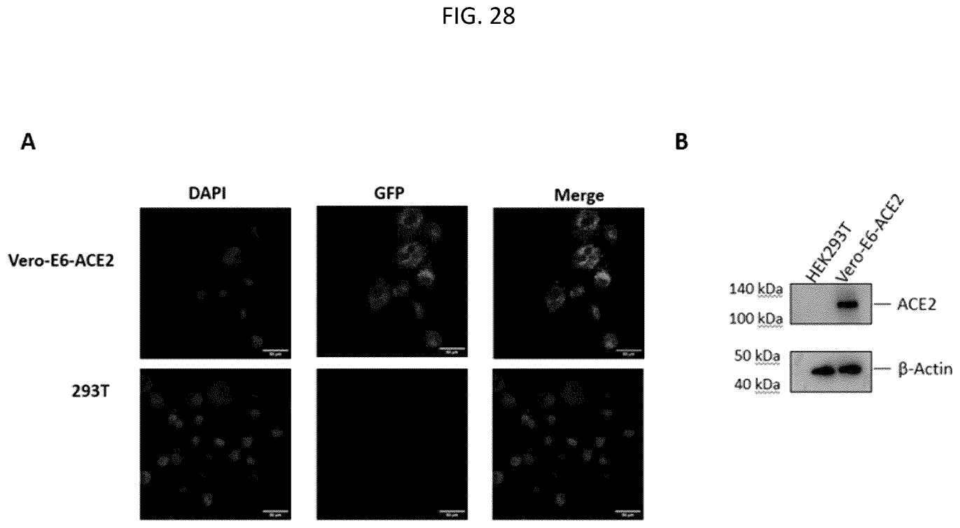

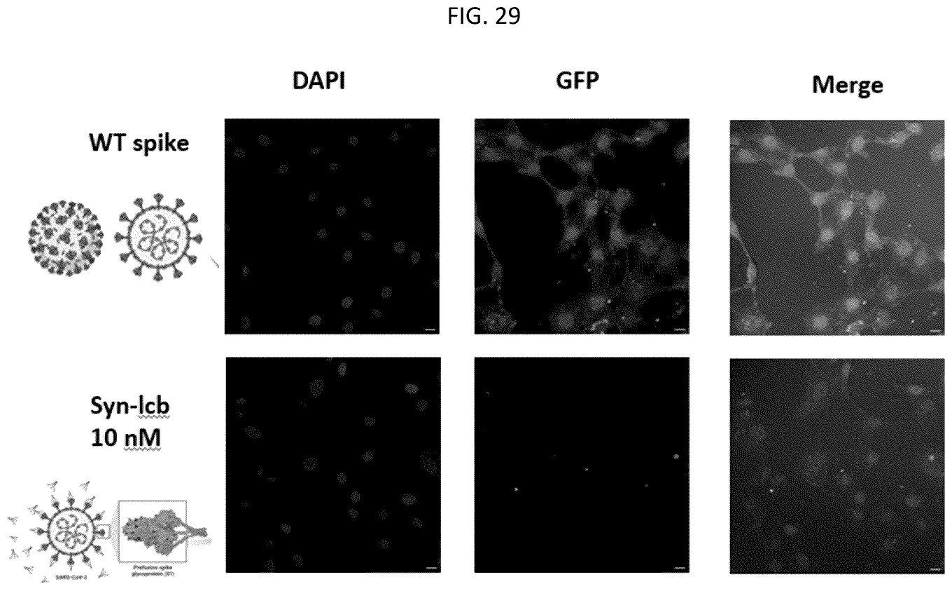

ACE is a key receptor protein for SARS-Cov-2 infestation of host cells. To detect the neutralizing effect of synbody on the pseudovirus, a stable Vero-E6 cell line expressing ACE2 was constructed. western blot results showed that Vero-E6-ACE2 highly expressed ACE2 protein, while 293T cells did not. Next, the GFP-tagged pseudovirus infected both Vero-E6-ACE2 and 293T cells, and after 48 h of infection, only the cells with ACE2 expression showed viral infection. (FIG. 28) Next, the pseudovirus was co-incubated with synbody for 30 min before infecting Vero-E6-ACE2 cells, while the same titer of viral infection was set as a control. The results showed that 10 nM of synbody was able to completely neutralize the viral infection. (FIG. 29)

### Tri-Bdbody Spike Molecular Structure

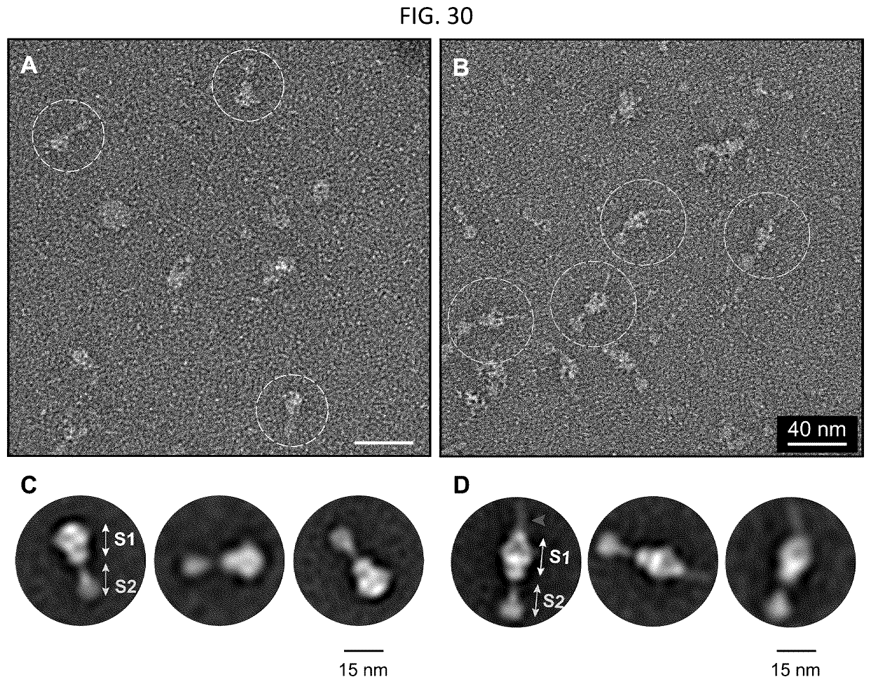

To confirm the molecular structure of spike with tri-pdbody, It shows synbody-spike protein (Delta variant) hybrid complex that was visualized using negative stain transmission electron microscopy (TEM) in FIG. 30. A freshly prepared 10 μl of sample was deposited on carbon B type copper grid (Ted Pella, part number 01814-F) that was pretreated in glow discharged for 1 min at 15 mA using a Pelco easiGlow-discharge system (Ted Pella, Redding, CA, USA). After 5 min, sample was wicked away and 5 μl of freshly prepared uranyl formate stain (2%) was applied to the grid. Then stain was blotted, and girds were dried in desiccator for 60 min prior to screening. All data were acquired on a FEI Tecnai TF20 TEM microscope operated at 120 kV accelerating voltage using a charge-coupled device (CCD) camera at 50000× magnification.

Panel A shows TEM image of full-length delta spike protein which are circled in white. Panel B shows synbody bound with spike protein which are circled in yellow. It confirmed that synbody can binds rigidly with spike protein S1 domain.

Moreover, class averaged images were generated using Relion3.0 software. Particles were manually picked, and class averaged (class 2D) without CTF correction. Panel C shows class 2D images of full-length delta spike protein. It can be clearly distinguished the head (S1 subunit) and stalk (S2 subunit) domain of full-length spike protein. Panel D shows the class 2D images generated from TEM images of synbody/spike protein hybrid complex. A rod shaped synbody structure can be clearly seen bound at S1 subunit of spike protein.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention. Several references are disclosed herein and each is incorporated herein by reference in its entirety.

We claim:

1. A DNA-peptide hybrid molecule comprising a DNA nanostructure chemically linked to one or more target-specific binding peptides, wherein the one or more target-specific binding peptides are specific for SARS-COV-2.

2. The composition of claim 1, wherein the DNA nanostructure is selected from the group consisting of: a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool.

3. The composition of claim 2, wherein the DNA nanostructure comprises one of a three-helix bundle or a four-helix bundle.

4. The composition claim 1, wherein the target-specific binding peptide is LCB1.

5. The composition of claim 1, wherein the distance between the target-specific binding peptides on the fully assembled DNA-peptide hybrid molecule is about the distance between the sites where the target-specific binding peptide binds to the native SARS-COV-2 homotrimeric spike protein.

6. The composition claim 1, wherein the DNA-peptide hybrid molecule has greater affinity for SARS-COV-2 spike protein compared to the one or more target-specific binding peptides alone.

7. The composition of claim 1, wherein the DNA-peptide hybrid molecule has a K_{d} of less than about 5 nM for each of the spike protein of SARS-COV-2 alpha, beta, gamma, and delta variants.

8. The composition of claim 1, wherein the synthetic DNA-peptide hybrid molecule further comprises an immunoglobulin Fc domain.

9. The composition of claim 8, wherein binding of the DNA-peptide hybrid molecule to SARS-COV-2 spike protein induces immune cells to engage in antibody dependent cellular cytotoxicity (ADCC).

10. The composition of claim 1, wherein the one or more target-specific binding peptides and the DNA nanostructure are linked through a disulfide bond or through copper-free click chemistry between a protein bearing the non-canonical amino acid 4-azidophenylalanine and DNA linked to cyclooctyne.

11. The composition of claim 1, wherein the chemical linkage of the one or more target-specific binding peptides to the DNA nanostructure is cleavable.

12. (canceled)

13. A method of treating a subject in need thereof, the method comprising administering to the subject a DNA-peptide hybrid molecule comprising a DNA nanostructure chemically linked to one or more target-specific binding peptides, wherein the one or more target-specific binding peptides are specific for SARS-COV-2, in an amount sufficient to treat SARS-CoV-2 infection.

14. The method of claim 13, wherein the DNA nanostructure is selected from the group consisting of: a three-helix bundle, a four-helix bundle, a six-helix bundle, a triangular DNA origami structure, a tetrahedral wireframe cage, a block-like origami cuboid, reconfigurable tweezers, double crossover tiles, branched three-way junctions, and a three-legged stool.

15. (canceled)

16. The method of claim 13, wherein the one or more target-specific binding peptides is LCB1.

17. The method of claim 13, wherein the distance between the target-specific binding peptides on the fully assembled DNA-peptide hybrid molecule is about the distance between the sites where the target-specific binding peptide binds to the native SARS-COV-2 homotrimeric spike protein.

18. The method of claim 13, wherein the DNA-peptide hybrid molecule has greater affinity for SARS-COV-2 spike protein than the target-specific peptide alone.

19. The method of claim 13, wherein the DNA-peptide hybrid molecule has a K_{d} of less than about 5 nM for each of the spike protein of SARS-COV-2 alpha, beta, gamma, and delta variants.

20. The method of claim 13, wherein the DNA-peptide hybrid molecule further comprises an immunoglobulin Fc domain.

21. (canceled)

22. The method of claim 13, wherein the one or more target-specific binding peptides and the DNA nanostructure are linked through a disulfide bond or through copper-free click chemistry between a peptide bearing the non-canonical amino acid 4-azidophenylalanine and DNA linked to cyclooctyne.

23. The method of claim 13, wherein the chemical linkage of the one or more target-specific binding peptides to the DNA nanostructure is cleavable.

24.-27. (canceled)


SEQUENCE LISTING (5 sequences)

SEQ ID NO: 1 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 56 aa, synthetic construct
DKEWILQKIYEIMRLLDELGHAEASMRVSDLIYEFMKKGDERLLEEAERLLEEVER

SEQ ID NO: 3 - protein, 6 aa, synthetic construct
  SITE 5..6: note = X= any amino acids
GPRPXX

SEQ ID NO: 4 - DNA, 14 bp, synthetic construct
  source 1..14: mol_type = other DNA
ctgaatggtacgga

SEQ ID NO: 5 - protein, 23 aa, synthetic construct
IEEQAKTFLDKFNHEADLFYQSC